SEQUENCE LISTING (22 sequences)

SEQ ID NO: 1 - RNA, 23 nt, synthetic construct
  source 1..23: mol_type = other RNA
  misc_feature 1..23: note = guide RNA
ccttagtgaagagcagtatgttc

SEQ ID NO: 2 - RNA, 23 nt, synthetic construct
  source 1..23: mol_type = other RNA
  misc_feature 1..23: note = guide RNA
aagagcagtatgttctgatctgg

SEQ ID NO: 3 - DNA, 20 bp, synthetic construct
  source 1..20: mol_type = other DNA
  misc_feature 1..20: note = PCR_primers_fwd_seq
cgtcgctcgctgagtgcctg

SEQ ID NO: 4 - DNA, 23 bp, synthetic construct
  source 1..23: mol_type = other DNA
  misc_feature 1..23: note = PCR_primers_rev_seq
tgtcgagtgtgaaagcgtcgagg

SEQ ID NO: 5 - DNA, 19 bp, synthetic construct
  source 1..19: mol_type = other DNA
  misc_feature 1..19: note = PCR_primers_fwd_seq
gagctttgcaccggcctat

SEQ ID NO: 6 - DNA, 21 bp, synthetic construct
  source 1..21: mol_type = other DNA
  misc_feature 1..21: note = PCR_primers_rev_seq
cttcatccagtcgaaggtccg

SEQ ID NO: 7 - DNA, 21 bp, synthetic construct
  source 1..21: mol_type = other DNA
  misc_feature 1..21: note = PCR_primers_fwd_seq
cctagcctacagggttctctc

SEQ ID NO: 8 - DNA, 22 bp, synthetic construct
  source 1..22: mol_type = other DNA
  misc_feature 1..22: note = PCR_primers_rev_seq
cacagagcgtactggtgaaaaa

SEQ ID NO: 9 - DNA, 20 bp, synthetic construct
  source 1..20: mol_type = other DNA
  misc_feature 1..20: note = PCR_primers_fwd_seq
acgtcagcaggttcgctatc

SEQ ID NO: 10 - DNA, 20 bp, synthetic construct
  source 1..20: mol_type = other DNA
  misc_feature 1..20: note = PCR_primers_rev_seq
agctgggctgtgactttgtt

SEQ ID NO: 11 - DNA, 19 bp, synthetic construct
  source 1..19: mol_type = other DNA
  misc_feature 1..19: note = PCR_primers_fwd_seq
agtcgagggtgcaggtatg

SEQ ID NO: 12 - DNA, 20 bp, synthetic construct
  source 1..20: mol_type = other DNA
  misc_feature 1..20: note = PCR_primers_rev_seq
ggccacttgttccactctct

SEQ ID NO: 13 - DNA, 19 bp, synthetic construct
  source 1..19: mol_type = other DNA
  misc_feature 1..19: note = PCR_primers_fwd_seq
caaacacagcatcgacggc

SEQ ID NO: 14 - DNA, 21 bp, synthetic construct
  source 1..21: mol_type = other DNA
  misc_feature 1..21: note = PCR_primers_rev_seq
cttcagtgggaggtcaggttc

SEQ ID NO: 15 - DNA, 19 bp, synthetic construct
  source 1..19: mol_type = other DNA
  misc_feature 1..19: note = PCR_primers_fwd_seq
tcatgctcaccagctccct

SEQ ID NO: 16 - DNA, 20 bp, synthetic construct
  source 1..20: mol_type = other DNA
  misc_feature 1..20: note = PCR_primers_rev_seq
cccccatagatctcgccaac

SEQ ID NO: 17 - DNA, 19 bp, synthetic construct
  source 1..19: mol_type = other DNA
  misc_feature 1..19: note = PCR_primers_fwd_seq
aagacttcacctaccgccc

SEQ ID NO: 18 - DNA, 19 bp, synthetic construct
  source 1..19: mol_type = other DNA
  misc_feature 1..19: note = PCR_primers_rev_seq
ggcgttgtgtggagcaagt

SEQ ID NO: 19 - DNA, 20 bp, synthetic construct
  source 1..20: mol_type = other DNA
  misc_feature 1..20: note = PCR_primers_fwd_seq
aaactcttcacggaccacgg

SEQ ID NO: 20 - DNA, 20 bp, synthetic construct
  source 1..20: mol_type = other DNA
  misc_feature 1..20: note = PCR_primers_rev_seq
ctcctgcttgcgaaacttgg

SEQ ID NO: 21 - DNA, 21 bp, synthetic construct
  source 1..21: mol_type = other DNA
  misc_feature 1..21: note = PCR_primers_fwd_seq
caagaaggtggtgaagcaggc

SEQ ID NO: 22 - DNA, 24 bp, synthetic construct
  source 1..24: mol_type = other DNA
  misc_feature 1..24: note = PCR_primers_rev_seq
gccaaattcgttgtcataccagga

(19) United States
(12) Patent Application Publication
(10) Pub. No.: US 2024/0425812 A1
(43) Pub. Date: Dec. 26, 2024

(54) HYPOVENTILATION PHYSICAL MODELS AND APPLICATIONS THEREOF

(71) Applicant: The University of Hong Kong, Hong Kong (HK)

(72) Inventors: Sau Wai Elly NGAN, Hong Kong (HK); Nga Chu LUI, Hong Kong (HK)

(21) Appl. No.: 18/748,156

(22) Filed: Jun. 20, 2024

Related U.S. Application Data
(60) Provisional application No. 63/509,033, filed on Jun. 20, 2023.

(51) Int. Cl. C12N 5/079; C12M 1/32; C12N 9/22

(52) U.S. Cl. CPC C12N 5/0618; C12M 23/12; C12N 9/22; C12N 2310/20; C12N 2501/13; C12N 2501/999; C12N 2503/02; C12N 2506/45; C12N 2513/00

(57) ABSTRACT

A hypoventilation physical model is provided. Particularly the physical model includes brainstem and cerebral organoids derived from pluripotent stem cells (PSCs) having a PHOX2B gene mutation. This mutation includes polyalanine repeat expansion mutations (PARMs), specifically 5-PARM, 7-PARM, and 13-PARM. These PARMs occur within a 20-alanine (20-Ala) polyalanine tract in exon 3 of the PHOX2B gene. The physical model provides a platform for studying impaired ventilatory responses and associated neuronal defects, offering a valuable tool for drug screening and the development of therapeutic strategies for hypoventilation syndromes.

### CROSS-REFERENCE TO RELATED APPLICATIONS

The present application claims priority from U.S. provisional patent application Ser. No. 63/509,033 filed Jun. 20, 2023, and the disclosure of which is incorporated herein by reference in its entirety.

### REFERENCE TO SEQUENCE DISCLOSURE

The sequence listing file under the file name “P2770US01_SEQ LISTING.xml” submitted in ST.26 XML file format with a file size of 29 KB created on Jun. 19, 2024 and filed on Jun. 20, 2024 is incorporated herein by reference.

### FIELD OF THE INVENTION

The present invention generally relates to the medical fields. More specifically the present invention relates to construct a hypoventilation physical model and provide a drug screening platform set for treating hypoventilation.

### BACKGROUND OF THE INVENTION

Ventilatory responses to hypoxia and/or hypercapnia are regulated by a specific set of chemoreceptors and motor neurons in the hindbrain. Among these, the retrotrapezoid nucleus (RTN) located at the ventral surface of the rostral medulla oblongata is a primary site for central chemoreceptors and respiratory drive. The RTN consists of a bilateral cluster of carbon dioxide (CO₂)-sensitive glutamatergic neurons that express vesicular-glutamate transporter-2 (VGLUT2) and the homeobox transcription factor PHOX2B. These glutamatergic neurons not only detect CO₂ levels and acid-base status in the brain but also integrate chemosensory information from peripheral chemoreceptors, such as blood gas composition detected by carotid oxygen (O₂)-sensitive chemoreceptors. As these RTN neurons exclusively innervate various regions of the ventrolateral medulla, which contains the respiratory pattern generator (RPG), they can stimulate breathing and enhance the rhythmic pattern of respiratory activity in response to elevated CO₂ levels. The RTN-mediated central respiratory chemoreflex provides an essential respiratory stimulus during episodes of apnea and dyspnea.

Congenital central hypoventilation syndrome (CCHS) is a life-threatening disease characterized by inadequate autonomic control of breathing, particularly during sleep. This condition results from an impaired ventilatory response to hypercarbia and hypoxemia due to the loss of RTN neurons in the brainstem. Polyalanine repeat expansion mutations (PARMs) in the PHOX2B gene, resulting in +5 to +13 extra alanines in the 20-residue polyalanine tract of PHOX2B, are strongly associated with CCHS. Patients with CCHS who have ≥+7Ala and +13Ala PARMs in PHOX2B often develop Hirschsprung disease (HSCR, characterized by the absence of enteric neurons in the distal colon) and neuroblastoma, respectively, indicating an impact on the peripheral nervous system (PNS) as well. The longer polyalanine repetitions are associated with aberrant cytoplasmic retention of PHOX2B, which adversely affects its transcriptional activity and its binding to the promoter of the dopamine beta-hydroxylase (DBH) gene. Thus, the length of PARMs in PHOX2B is closely related to the clinical phenotypes and disease severity observed in patients.

Mouse mutants carrying +7Ala PARM in Phox2b lack Phox2b+RTN neurons but exhibit an enrichment of Phox2b-expressing glutamatergic neurons on the dorsal side of the facial nucleus (nVII). These mutants display CCHS-like phenotypes, including breathing defects, an absent response to hypercapnia, and central apnea leading to death shortly after birth. In contrast, Phox2b null mutants exhibit more severe defects in both the central (CNS) and peripheral nervous systems. These null mutants lack visceral and branchial motor neurons in the hindbrain and fail to form the sympathetic, parasympathetic, and enteric ganglia in the PNS. Compared to other neurons in the CNS/PNS, RTN neurons appear to be most vulnerable to PHOX2B-PARMs, or PHOX2B-PARMs may specifically disrupt the expression of genes crucial for RTN neuron formation. To date, only a few mouse models have successfully recapitulated CCHS-like phenotypes. It remains largely unclear how PARMs alter PHOX2B functions and interfere with RTN neuron formation in the brainstem and whether longer polyalanine repetitions in PHOX2B lead to more severe CNS defects and disrupt the development of neural crest derivatives for the enteric nervous system (ENS), contributing to CCHS and associated ENS defects.

However, there is no well-established human model of CCHS available for drug screening, significantly hindering drug development efforts. The critical nature of CCHS underscores the urgent need for screening and identifying effective treatments for this condition. Therefore, the present invention addresses this need.

### SUMMARY OF THE INVENTION

It is an objective of the present invention to provide models, systems, or method to solve the aforementioned technical problems.

In accordance with a first aspect of the present invention, a hypoventilation physical model is provided. The physical model includes a brainstem organoid and a cerebral organoid, wherein both of the brainstem organoid and the cerebral organoid are formed by pluripotent stem cells (PSCs) having a PHOX2B gene mutation.

In accordance with one embodiment of the present invention, the PHOX2B gene mutation includes a polyalanine repeat expansion mutation (PARM).

In accordance with one embodiment of the present invention, the PARM includes a 5-PARM, a 7-PARM and a 13-PARM.

In accordance with one embodiment of the present invention, the PARM is happened on a 20-Ala polyalanine tract in exon 3 of the PHOX2B gene.

In accordance with one embodiment of the present invention, the brainstem organoid is fabricated with the following steps:
suspending the PSCs into a single cell suspension;
seeding the single cell suspension to a microwell plate with a density of 8,000-10,000 cells per microwell and incubating them overnight to form spheroids;
transferring the spheroids to a low-attachment plate comprising a first culture medium supplemented with a BMP inhibitor and a TGF-β inhibitor in a ratio of approximately 1:8 to 1:12 followed by culturing;
changing the medium to a second culture medium supplemented with the BMP inhibitor, TGF-β inhibitor, and a SHH agonist followed by culturing under agitation;
changing the medium to the second culture medium supplemented with the SHH agonist, a WNT agonist and retinoic acid (RA) followed by incubation;
changing the medium to a third culture medium supplemented with BDNF, GDNF, dibutyryl-cAMP and an acid for incubation; and
collecting matured brainstem organoids.

In accordance with one embodiment of the present invention, the cerebral organoid is generated by the following steps:
suspending the PSCs in an embryoid body formation medium with ROCK inhibitor to form a single cell suspension;
transferring the PSCs to a low-attachment plate for culturing for 6-14 hours to form embryoid bodies;
further culturing the embryoid bodies in the embryoid body formation medium for 36-56 hours;
transferring the embryoid bodies to an induction medium for an incubation of 36-60 hours to induce the formation of neuroepithelia;
embedding the embryoid bodies in a basement membrane matrix for further expansion;
changing the medium to a maturation medium for culturing under agitation; and
collecting matured cerebral organoids.

In accordance with one embodiment of the present invention, the brainstem organoid includes RTN neurons and the brainstem organoid exhibits increased firing in response to elevated CO₂ levels.

In accordance with one embodiment of the present invention, the cerebral organoid includes functional neurons expressing glutamatergic, GABAergic, cholinergic, and serotonergic markers.

In accordance with one embodiment of the present invention, it further includes a neuronal differentiation medium optimized for producing enteric nervous system (ENS) neurons.

In accordance with a second aspect of the present invention, a system for screening a potential drug candidate for treating hypoventilation based on ventilatory response CO₂ changes is provided. Particularly, the system includes the aforementioned hypoventilation physical model for mimicking the impaired ventilatory response to hypercarbia and hypoxemia.

In accordance with a third aspect of the present invention, a method of treating hypoventilation is introduced. Specifically, the method includes identifying a candidate drug using the aforementioned system and administering an effective amount of the candidate drug to a subject in need thereof.

In accordance with a fourth aspect of the present invention, a hypoventilation physical model including a brainstem organoid and a cerebral organoid is provided. Particularly, the brainstem organoid includes PHOX2B+ neurons with reduced dopamine beta-hydroxylase (DBH) expression.

In accordance with one embodiment of the present invention, the PHOX2B gene mutation is corrected using CRISPR/Cas9 genome editing to increase the yield of DBH+ neurons.

In accordance with one embodiment of the present invention, the PHOX2B gene mutation is introduced via genome editing techniques.

In accordance with one embodiment of the present invention, the modul further includes peripheral neurons derived from human enteric neural crest cells (hENCCs).

In accordance with one embodiment of the present invention, the hENCCs are differentiated to express PHOX2B and exhibit ENS-associated defects.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

Embodiments of the invention are described in more details hereinafter with reference to the drawings, in which:

FIGS. 1A-IK depicts generation of brainstem organoids (HBSOs) comprising hypercapnia-responsive neurons; FIG. 1A is a schematic diagram showing the derivation of HBSOs from hPSCs, in which representative phase-contrast images showing the HSBOs derived from the control hPSCs at different stages of differentiation; FIG. 1B is a schematic diagram showing the rostro-caudal pattern of neural tube, in which the bar charts showing the RT-qPCR analyses of the relative expressions of HOXA2, HOXB1, HOXB2, GBX2 and OTX2 in the day 20 control HBSOs in comparison to the day 60 HCOs; FIG. 1C is a schematic diagram showing the dorso-ventral pattern of neural tube, in which the bar charts showing the RT-qPCR analyses of the relative expressions of PAX7, OLIG3 and NGN in the day 20 control HBSOs in comparison to the day 60 HCOs; FIG. 1D is a bar chart showing the RT-qPCR analyses of the relative expression of PHOX2B in the day 20 control HBSOs in comparison to the day 60 HCOs; FIG. 1E shows the immunostaining of PHOX2B and markers of forebrain (TBR1), midbrain (FOXA2, OTX2) and hindbrain (ISL1) in the day 60 control HBSOs; FIG. 1F shows a schematic diagram on the left indicating the different domains and their markers found in the respiratory center of the medulla in vivo and the immunostaining of PHOX2B, TH, VGLUT2 and 5-HT in the day 60 control HBSOs on the right, in which the inset on the top-right corner is an enlargement of a square region in the image and the arrowhead marks the RTN-like (PHOX2B+VGLUT2+TH-) neurons; FIG. 1G shows the MEA analysis of day 60 control HBSO neurons in response to different CO₂ levels (FIG. 1H), RTN blocker (KCNQ channel activator, retigabine) (FIG. 11), 5-HT (FIG. 1J) and 5-HT channel blocker (SB269970) (FIG. 1K);

FIGS. 2A-2H depicts the high-resolution scRNA-seq analyses revealing transcriptome heterogeneity and regional characteristics of HBSO cells; FIG. 2A shows the UMAP projection of all 23,088 individual cells from day 60 HBSOs, colored by cell types; FIG. 2B is a lineage cladogram showing the hierarchical relationship of the cell types based on the average expression of top 1,500 variable features; FIG. 2C exhibits violin plots showing the key markers of six main cell types; FIG. 2D exhibits violin plots showing the key markers of six neuronal subtypes; FIG. 2E shows the expression of regional marker HOXA2, HOXB2 and NKX2-2 in HBSO cells; FIG. 2F shows the pathway scores of hindbrain development in HCOs and HBSOs; FIG. 2G depicts the expression of regional markers TOX3 (r5) and PRDX6 (r6) in HCOs and HBSOs; FIG. 2H shows the similarities between different organoids estimated by Spearman correlation;

FIGS. 3A-3J shows the detrimental effect of PHOX2B-7Ala in generation of the RTN-like neurons in HBSOs; FIG. 3A depicts the representative phase-contrast images of the day 60 HBSOs derived from the control and the PHOX2B-7Ala mutant; FIG. 3B shows the immunostaining of PHOX2B, 5-HT in the day 60 control and PHOX2B-7Ala HBSOs; FIG. 3C shows the immunostaining of PHOX2B, TH and VGLUT2 in the day 60 control and PHOX2B-7Ala HBSOs, in which the inset on the bottom is an enlargement of a square region in the image and the filled and open arrow heads mark the RTN-like (PHOX2B+VGLUT2+TH−) and PHOX2B+TH+ neurons, respectively; FIG. 3D shows the Western blot of PHOX2B expression in the day 60 control and PHOX2B-7Ala HBSOs; FIG. 3E shows the RT-qPCR analyses of PHOX2B expression in the day 60 control and PHOX2B-7Ala HBSOs; FIG. 3F is a bar chart showing the percentages of PHOX2B+ cells in the day 60 HBSOs derived from the control and PHOX2B-PARM mutants; FIG. 3G is a bar chart showing the percentages of PHOX2B⁺VGLUT2⁺ cells in the day 60 HBSOs derived from the control and PHOX2B-PARM mutants; FIG. 3H is a bar chart showing the percentages of PHOX2B⁺TH⁺ cells in the day 60 HBSOs derived from the control and PHOX2B-PARM mutants; FIGS. 3I and 3J show the immunostaining of PHOX2B with C-Casp3 and TUJI (an enlargement of a square region in the image is shown in the right panel) in the day 60 control and PHOX2B-7Ala HBSOs, respectively;

FIGS. 4A-4F shows that the PARMs in PHOX2B interferes the formation of HBSOs; FIG. 4A shows the RNA velocity analyses revealing the differentiation paths of various neuronal subtypes are perturbed in PHOX2B-7Ala HBSOs; FIG. 4B is a barplot showing the population size difference of six neuronal subtypes between control and PHOX2B-7Ala mutant, in which the disrupted populations size in PHOX2B-7Ala mutant are listed on the bottom table; FIG. 4C is a Barplot showing the disrupted pathways in pooled neuronal cells in PHOX2B-7Ala HBSOs; FIG. 4D depicts the violin plots showing the down-regulation of key genes involved in neuronal differentiation (upper panel) and hindbrain regionalization (lower panel) in PHOX2B-7Ala mutant; FIGS. 4E and 4F show the immunostaining of PHOX2B and ISL1 and NKX2-2 in the day 60 control and PHOX2B-7Ala HBSOs, respectively;

FIGS. 5A-5F shows that the PARMs in PHOX2B disrupt the formation of respiratory center-like region; FIG. 5A is a Schematic diagram showing the additional alanine (Ala) repeats added to the PHOX2B-PARM mutant hPSC lines; FIG. 5B shows the immunostaining of different markers of the respiratory center-like region in the day 60 control HCOs, in which the inset on the bottom is an enlargement of a square region in the image and yellow arrows mark the RTN-like (PHOX2B⁺ VGLUT2⁺TH⁻) neurons; FIG. 5C shows the representative phase-contrast images of day 60 HCOs derived from the control and the PHOX2B-PARM mutants; FIG. 5D depicts the immunostaining of markers of A5 domain (PHOX2B⁺; TH⁺; VGLUT2⁻), C1 domain (PHOX2B⁺; TH⁺; VGLUT2⁺) and RTN neurons (PHOX2B⁺; TH⁻; VGLUT2⁺) in the day 60 HCOs derived from the control and the PHOX2B-PARM mutants, in which the yellow arrows mark the RTN-like (PHOX2B⁺VGLUT2⁺TH⁻) neurons; FIGS. 5E and 5F are bar charts showing the percentages of PHOX2B⁺ and PHOX2B⁺; VGLUT2+double-positive cells in respiratory center-like region of the day 60 HCOs derived from the control and PHOX2B-PARM mutants, respectively;

FIGS. 6A-6M shows the scRNA-seq analysis of HCOs revealing that PARM in PHOX2B interrupts the progenitor-to-neuronal transition of PHOX2B⁺ cells; FIG. 6A depicts the t-SNE projection of all 72,800 individual cells from day 60 HCOs; FIG. 6B depicts that there are 16 clusters classified into 6 main cell types in t-SNE projection; FIG. 6C depicts the PHOX2B expressing cells in the control HCOs after imputation analysis; FIG. 6D depicts the trajectory constructed by PCA analysis showing that PHOX2B⁺ cells progress from NSC to IP and CN states in control HCOs; FIG. 6E depicts the canonical markers expressing in PHOX2B⁺ cells at NSC, IP and CN states in control HCOs; FIG. 6F shows the transcription factor (TF) correlation network controlled the lineage progression from NSC to IP and CN states, in which each node indicates a TF and the edge represents the high correlation (Spearman's rank correlation coefficient >0.3); FIG. 6G depicts the immunofluorescence showing the co-expression of Phox2b with NSC/IP (Sox2), CN (NeuroD6) and midbrain (Otx2) markers during embryonic mouse brain development as marked by the arrowheads; FIG. 6H shows the PHOX2B⁺ cells in PHOX2B-7Ala HCOs; FIG. 6I depicts the trajectory constructed by PCA analysis showing the lineage progression of PHOX2B⁺ cells from NSC to IP and CN states in PHOX2B-7Ala HCOs; FIG. 6J depicts the canonical markers expressed in PHOX2B⁺ cells at NSC, IP and CN states in PHOX2B-7Ala HCOs; FIG. 6K is a bar plot showing the population sizes of PHOX2B⁺ cells at NSC, IP and CN states in the control and PHOX2B-7Ala HCOs;

FIGS. 6L and 6M respectively show the immunostaining of PHOX2B⁺; SOX2⁺ and PHOX2B⁺; NEUROD6⁺ expressing cells in the control and PHOX2B-7Ala mutant HCOs as marked by the arrowheads, in which an enlargement of a square region in the image is shown in the right panel;

FIGS. 7A-7J demonstrates that the PARM in PHOX2B interrupts the pattern specification of PHOX2B⁺ neurons in HCOs; FIG. 7A depicts the immunostaining showing the nuclear localization of PHOX2B proteins in the day 60 control and PHOX2B-7Ala mutant HCOs; FIG. 7B shows the GRN activity of PHOX2B in the control and mutant HCOs; FIG. 7C depicts the violin plots showing the expression of PHOX2B hindbrain targets in the control and mutant CN; FIG. 7D is a scatter plot showing putative PHOX2B-7Ala activated (red) and repressed (blue) targeted genes; FIG. 7E depicts the gene ontology (GO) enrichment analyses of PHOX2B activated and repressed targeted genes; FIG. 7F shows the overall pathway scores of embryonic organ morphogenesis and pattern; FIG. 7G depicts the violin plots showing the expression of HOX genes in the control and mutant CN; FIG. 7H depicts the violin plots showing the expression of Hedgehog pathway genes in the control and mutant CN; FIG. 7I shows the immunostaining of PHOX2B and markers of forebrain, midbrain and hindbrain (TBR1, FOXA2 and ISL1) in the day 60 PHOX2B-7Ala HCOs, in which an enlargement of a square region in the image is shown in the bottom; and FIG. 7J shows the immunostaining of PHOX2B and OTX2 in the day 60 control and PHOX2B-7Ala HCOs, in which an enlargement of a square region in the image is shown in the right panel;

FIGS. 8A-8M depicts the hPSC-based models of CCHS-HSCR revealing differential vulnerability of RTN and ENS neurons to PARM; FIG. 8A is a schematic diagram showing the use of hPSC-based models of CCHS-HSCR to evaluate the effect of PHOX2B-PARMs in CNS and ENS neurons; FIG. 8B depicts the electrographs of Sanger sequencing of PHOX2B exon 3 reveal the “correction” of +7Ala PARM in PHOX2B locus; FIG. 8C depicts the representative phase-contrast images of day 60 HBSOs derived from 15C11 and 15C11-C hPSC lines and the bar chart showing the size of the day 60 HBSOs; FIG. 8D depicts the immunostaining of PHOX2B and ISL1 in the day 60 15C11 and 15C11-C HBSOs, in which the arrowheads mark the PHOX2B⁺ISL1⁺ neuron; FIG. 8E depicts the immunostaining of PHOX2B, TH and VGLUT2 in the day 60 15C11 and 15C11-C HBSOs, in which the putative RTN (PHOX2B⁺VGLUT2⁺TH⁻) and PHOX2B⁺TH⁺ neurons are marked by filled and open arrowheads, respectively; FIGS. 8F-8H are bar charts showing the percentages of PHOX2B⁺, PHOX2B⁺VGLUT2+ and PHOX2B⁺TH⁺ cells in the day 60 15C11 and 15C11-C HBSOs, respectively; FIGS. 8I and 8J depict the immunostaining of PHOX2B and DBH or TH, respectively, in the day 40 ENS neurons derived from the control, PHOX2B-PARM mutants, 15C11 and 15C11-C lines; FIGS. 8K-8M are the bar charts showing the percentages of PHOX2B⁺, DBH⁺ and TH⁺ ENS neurons, respectively, in the different groups;

FIGS. 9A-9B depict the quality control and reference-based annotation of HBSO cells; FIG. 9A shows the number of detected gene and UMI number, and percentage of mitochondrial genes between control and PHOX2B-7Ala mutant HBSOs; FIG. 9B shows the distribution of the six main cell types of in vivo mouse hindbrain cells from E12-E16 and the pooled HBSOs of the present invention in UMPA plot;

FIG. 10 demonstrates the targeting strategy for introduction and correction of PARM-mutations in human PHOX2B allele;

FIGS. 11A-11B exhibit the cell composition in control and PHOX2B-7Ala mutant HBSOs as revealed by scRNAseq; FIG. 11A shows the distribution of the six main cell types of the control and PHOX2B-7Ala mutant HBSOs in a new UMAP plot; FIG. 11B is a bar plot showing the population size difference of six main cell types between control and PHOX2B-7Ala mutant;

FIG. 12 is a schematic diagram showing the derivation of HCOs from hPSCs;

FIGS. 13A-13B depicts the respiratory center-like region in day 60 HCO; FIG. 13A shows the representative images of day 60 HCOs derived from the control and PHOX2B-PARM mutants, in which the HCOs are stained with PHOX2B and 5-HT, representing the respiratory center-like regions and the inset on the bottom is an enlargement of the white squared region in the image; FIG. 13B is a bar chart showing the percentage area of respiratory center-like region in the control and PHOX2B-PARM mutant HCOs;

FIGS. 14A-14D show quality control and overall characteristics of single-cell RNA-seq dataset; FIG. 14A shows the number of detected UMI and gene numbers in four HCO samples; FIG. 14B depicts the distribution of the 6 clusters from the control and PHOX2B-7Ala mutant HCOs in t-SNE plot (colored by clusters); FIG. 14C exhibits the canonical markers expressed in 16 clusters in the integrated HCO cells, colored by expression level; and FIG. 14D is a heatmap showing the unique markers of each cluster;

FIGS. 15A-15D depict the fine annotations of 16 clusters into 6 functional cell types and imputation analysis; FIG. 15A is a heatmap showing the unique markers of 6 main cell types in the day 60 HCOs; FIG. 15B is a dot-pot showing the top key markers expressed in each cell type; FIG. 15C shows the expression of PHOX2B before imputation; and FIG. 15D shows the expression of PHOX2B after imputation; and

FIGS. 16A-16C demonstrate the establishment and characterization of CCHS-HSCR-hPSC (15C11) line; FIG. 16A shows the karyotype of 15C11 hPSC; FIG. 16B depicts that the promoter of NANOG is unmethylated in the 15C11 hPSC; FIG. 16C shows the immunocytochemistry results indicating the expression of stem cell markers in the 15C11 hPSC.

### DETAILED DESCRIPTION

In the following description, models, systems, and methods of screening a potential drug candidate for treating hypoventilation and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

Organoids are three-dimensional (3D) cell cultures derived from stem cells that mimic the structure and function of real organs. They are grown in vitro and are used as models for studying development, disease, and potential treatments. The key features of organoids include their ability to self-organize. Stem cells in organoids can differentiate and self-organize into structures that resemble the architecture of actual organs. Organoids can perform some of the functions of the organs they model, providing a more accurate representation than traditional 2D cell cultures.

Cerebral organoids are derived from human pluripotent stem cells (hPSCs) and are used to model early brain development. They can mimic the complex structure and cellular diversity of the human brain, including regions like the cerebral cortex. They are used to study neurological development, brain disorders (such as microcephaly, autism, and Alzheimer's disease), and the effects of drugs on brain cells.

Brainstem organoids are designed to model the brainstem, the part of the brain that connects the cerebrum with the spinal cord and controls vital functions such as breathing, heart rate, and sleep cycles. Brainstem organoids are also developed from stem cells and require specific differentiation protocols to generate the appropriate cell types and structures.

However, the combination of these two types of organoids is required in order to adequately replicate human physiological and genetic conditions associated with hypoventilation syndromes. A hypoventilation physical model according to the present invention includes both brainstem organoids and cerebral organoids, both derived from pluripotent stem cells (PSCs) containing a specific mutation in the PHOX2B gene. The PHOX2B gene mutation of interest includes polyalanine repeat expansion mutations (PARMs), particularly 5-PARM, 7-PARM, and 13-PARM. These mutations occur within a 20-alanine polyalanine tract in exon 3 of the PHOX2B gene.

To fabricate the brainstem organoid, PSCs are first suspended into a single cell suspension. This suspension is seeded onto a microwell plate at a density of 8,000-10,000 cells per microwell and incubated overnight at 37° C. to form spheroids. These spheroids are then transferred to a low-attachment plate containing a first culture medium, which includes a BMP inhibitor at a concentration of 1 μM and a TGF-β inhibitor at 10 μM, and cultured for two days. Following this, the medium is changed to a second medium supplemented with the BMP inhibitor, TGF-β inhibitor, SHH agonist, and WNT antagonist IWR1-endo, and the culture is maintained under shaking conditions at 90 r.p.m for three days. Subsequently, the medium is again changed to the second medium including the SHH agonist, a WNT agonist, and retinoic acid (RA) for an additional six days of incubation. Finally, the medium is switched to a third culture medium containing 20 ng/ml BDNF, 20 ng/ml GDNF, 50 nM dibutyryl-cAMP, and 200 μM ascorbic acid for a prolonged incubation of 48 days. The matured brainstem organoids, which exhibit increased firing in response to elevated CO₂ levels and comprise retrotrapezoid nucleus (RTN) neurons, are then collected.

In one embodiment, the first medium is a neural induction medium composed of Dulbecco's modified eagle medium/nutrient mixture F-12 (DMEM/F12) supplemented with 1% N2, 1% GlutaMAX, 1% MEM-NEAA, and 1 μg/ml heparin.

In one embodiment, the second medium is a neural differentiation medium composed of a 1:1 mixture of DMEM/F12 and neurobasal medium with 0.5% N2 supplement (v/v), 1% B27 supplement without Vitamin A (v/v), 1% GlutaMAX (v/v), 0.5% MEM non-essential amino acids (NEAA), 2.5 μg ml⁻¹ insulin, 49.5 μM β-mercaptoethanol and 1% Penicillin-Streptomycin (Pen-Strep) (v/v).

For the generation of cerebral organoids, PSCs are suspended in an embryoid body formation medium containing a ROCK inhibitor to form a single cell suspension. These cells are transferred to a low-attachment plate and cultured overnight at 37° C. to form embryoid bodies. The embryoid bodies are maintained in the formation medium for two days and then transferred to an induction medium for two more days to promote the formation of neuroepithelia. These embryoid bodies are then embedded in a basement membrane matrix for further expansion, and the medium is changed to a maturation medium for a 49-day culture period under shaking conditions at 75 r.p.m. The resulting matured cerebral organoids, which contain functional neurons expressing glutamatergic, GABAergic, cholinergic, and serotonergic markers, are subsequently collected.

This physical model also incorporates a neuronal differentiation medium optimized for producing enteric nervous system (ENS) neurons, further broadening its applicability in studying diverse neuronal populations.

Overall, this hypoventilation physical model provides a valuable platform for investigating the genetic and physiological mechanisms underlying hypoventilation syndromes, facilitating drug screening and the development of therapeutic strategies targeting these conditions

In accordance with a second aspect of the present invention, a system for screening a potential drug candidate for treating hypoventilation based on ventilatory response CO₂ changes is provided. Particularly, the system includes the aforementioned hypoventilation physical model for mimicking the impaired ventilatory response to hypercarbia and hypoxemia.

In accordance with a third aspect of the present invention, a method of treating hypoventilation is introduced. Specifically, the method includes identifying a candidate drug using the aforementioned system and administering an effective amount of the candidate drug to a subject in need thereof.

In accordance with a fourth aspect of the present invention, a sophisticated hypoventilation physical model including both brainstem and cerebral organoids is provided. This physical model is specifically designed to replicate the genetic and physiological characteristics associated with hypoventilation syndromes. Notably, the brainstem organoid includes PHOX2B+ neurons that exhibit reduced expression of dopamine beta-hydroxylase (DBH), a key enzyme involved in catecholamine synthesis. This physical model leverages pluripotent stem cells (PSCs) with a mutation in the PHOX2B gene to faithfully mimic the disease state.

In one embodiment, the PHOX2B gene mutation is corrected using the CRISPR/Cas9 genome editing system, which has been shown to significantly increase the yield of DBH+ neurons in the brainstem organoid. This correction not only enhances the physical model's accuracy in representing normal physiological conditions but also provides a powerful tool for studying the underlying mechanisms of hypoventilation syndromes and potential therapeutic interventions.

The introduction of the PHOX2B gene mutation in this model is achieved through advanced genome editing techniques. This approach ensures precise and targeted modifications in the genetic sequence, allowing for the creation of organoids that closely replicate the genetic anomalies observed in patients with hypoventilation syndromes.

Furthermore, the hypoventilation physical model also incorporates peripheral neurons derived from human enteric neural crest cells (hENCCs). These hENCCs are differentiated to express PHOX2B and exhibit defects associated with the enteric nervous system (ENS). This addition enriches the physical model by providing a comprehensive view of the ENS-associated abnormalities that are often linked with hypoventilation disorders.

Through these detailed methodologies, the hypoventilation physical model offers an invaluable platform for investigating the complex interactions between different neuronal populations and the genetic mutations implicated in hypoventilation syndromes. It serves as a robust system for screening potential drug candidates and developing targeted therapies aimed at alleviating the symptoms and underlying causes of these debilitating conditions.

### EXAMPLES

### Plasmid Constructions

The human codon-optimized Cas9 expression plasmid is obtained from Addgene (44720). Two guide RNAs (gRNA) are designed using the CRISPR design tool to target the 20-Ala polyalanine tract in exon 3 of PHOX2B, which are PHOX2B-gRNAa with a sequence of SEQ ID NO: 01 (5′-CCTTAGTGAAGAGCAGTATGTTC-3′) and PHOX2B-gRNAb having a sequence of SEQ ID NO: 02 (5′-AAGAGCAGTATGTTCTGATCTGG-3′). The gRNA plasmid (Addgene, 41824) is linearized with AflII and the gRNA targeting sequence is cloned into gRNA plasmid using Gibson Assembly (New England Biolabs, E2611) according to Church's lab protocol.

As used herein, the term of “20-Ala polyalanine tract” refers to a specific sequence within the PHOX2B gene that encodes a stretch of 20 consecutive alanine (Ala) residues in the resulting PHOX2B protein. This tract is located within an exon of the PHOX2B gene and results in a segment of the protein composed entirely of alanine amino acids. Polyalanine tracts can influence the structural and functional properties of the protein. In the case of PHOX2B, mutations leading to expansions of this polyalanine tract are associated with congenital central hypoventilation syndrome (CCHS), affecting the protein's normal function and localization.

The donor plasmid, pHR-PHOX2B-L-2A-EGFP, containing the homology arms spanning a region from chr4: 41746593-41745806 (GRCh38.p13) and a puromycin selection cassette designed for PHOX2B targeting, is a generous gift from Dr. Yohan Oh (Johns Hopkins University School of Medicine, Baltimore). For construction of PHOX2B-PARM mutant donor plasmids containing different length of PARMs, oligos with additional Ala repeats are annealed and extended using Q5 High-fidelity DNA Polymerase (New England Biolabs, M0491). Annealed double-stranded oligos containing the desired additional Ala repeats (+5Ala, +7Ala and +13Ala) together with a 189-bp PCR fragment containing the 3′-end of the polyalanine tract region are subcloned into the ˜8 kb PCR fragment of the wild-type PHOX2B donor plasmid backbone (all designed with ˜40 bp overlapping sequences at each end) by Gilson Assembly according to manufacturer's protocol. DNA sequence of all the PHOX2B-PARM mutant donor plasmids is confirmed by Sanger sequencing.

Generation of PHOX2B-PARM Mutant hPSC Cell Lines

The CRISPR-Cas9 system is used to target the exon3 of PHOX2B allele (after stop codon) in the UE02302 control hPSCs. The hPSCs are transfected with the mutant PHOX2B-PARM donor plasmids together with the gRNA constructs and a Cas9 nickase expression plasmid using Human Stem Cell Nucleofector Kit 2 (Lonza, VPH-5022). The transfected cells are selected for 7 days in 0.2 μg ml⁻¹ Puromycin and the Puromycin-resistant clones are isolated and passaged twice before genotyping. The targeted region of PHOX2B gene is genotyped to confirm the site-specific insertion of PARMs using Sanger sequencing. The mutant hPSC clones with the confirmed PARM insertion are expanded for further assays.

Generation of Brainstem Organoids (HSBOs) from hPSCs

To generate HSBOs, hPSCs are dissociated into single cell suspension by treating with 0.5 mM EDTA solution in DPBS and then Accutase as described above. 2.7×10⁶ cells are seeded to a well of AggreWell800 24-well plate (STEMCELL Technologies, 34815) which contained 300 microwells per well (i.e. 9,000 cells per microwell). The cells are kept in mTeSR1 with 10 μM Y-27632 overnight in 37° C. incubator for the spheroid formation and they are marked as day 0 at this stage. At day 1, the spheroids from one well of Aggre Well800 are split into 3 wells of 6-well ultra-low-attachment plate containing Neural Induction Medium (DMEM/F12, 1% N2 supplement (v/v), 1% GlutaMAX supplement (v/v), 1% MEM-NEAA (v/v) and 1 μg ml⁻¹ Heparin) supplemented with BMP inhibitor LDN-193189 (1 μM) and TGF-β inhibitor SB431542 (10 μM). From day 3 to 6, the organoids are incubated in Neural Differentiation Medium (NDM) containing DMEM/F12 and Neurobasal Medium in 1:1 ratio with 0.5% N2 supplement (v/v), 1% B27 supplement without Vitamin A (v/v), 1% GlutaMAX (v/v), 0.5% MEM non-essential amino acids (NEAA), 2.5 μg ml⁻¹ insulin, 49.5 μM β-mercaptoethanol and 1% Penicillin-Streptomycin (Pen-Strep) (v/v). LDN-193189 (1 μM), SB431542 (10 μM), SHH agonist SAG (1 μM) and different concentrations (0, 1 and 10 μM) of WNT antagonist IWR1-endo are added to NVM at day 3 to 6. Starting from day 3, the organoids are kept shaking at 90 r.p.m. with an orbital shaker installed inside the incubator. From day 6 to 12, the organoids are maintained in NDM with SAG (1 μM), WNT agonist CHIR99021 (3 μM) and retinoic acid (RA) (1 μM) and the medium is changed every 2-3 days. From day 12 onwards, the organoids are maintained in Neural Maturation Medium (NMM) (same recipe as NDM but with B27 containing Vitamin A) supplemented with 20 ng ml⁻¹ BDNF, 20 ng ml⁻¹ GDNF, 50 nM dibutyryl-cAMP and 200 μM ascorbic acid. The medium is changed every 3-4 days until day 60, when the organoids are collected.

Generation of Human Cerebral Organoids (HCOs) from hPSCs

HCOs are generated from hPSCs using a commercially optimized kit, STEMdiff Cerebral Organoid Kit (STEMCELL Technologies, 08570) following the manufacturer's protocol and previously described protocols with modifications. First, to generate embryoid body (EB) from hPSCs for the formation of HCOs, hPSCs are washed with DPBS (Cytiva, SH30028.03) and treated with 0.5 mM EDTA solution in DPBS for 4 min at 37° C. After removing the EDTA solution, the cells are dissociated into single cell suspension by treating with Accutase (STEMCELL Technologies, 07922) for 4 min at 37° C. The cells are pelleted by centrifugation for 5 minutes at 270×g at room temperature and then resuspended with EB Formation Medium with ROCK inhibitor Y-27632 (10 μM). 9,000 cells are transferred to each well of 96-well round bottom ultra-low attachment plate (Corning, 7007) and kept undisturbed overnight in 37° C. incubator for the EB formation. The cells are marked as day 0 at this stage. At day 2 and 4, the EBs are fed with additional 100 μl EB Formation Medium per well. On day 5, one EB is transferred to each well of 24-well ultra-low attachment plate (Corning, 3473) with 500 μl Induction Medium to induce the formation of neuroepithelia. On day 7, the organoids are embedded in Matrigel and transferred to 6-well ultra-low attachment plate (Corning, 3471) for further expansion in Expansion Medium. On day 11, the medium is replaced with Maturation Medium and the 6-well plate (with 6-8 organoids per well) is kept shaking at 75 r.p.m. with an orbital shaker installed inside the incubator. The medium is changed every 3-4 days until day 60, when the organoids are collected.

Fluorescence-Activated Cell Sorting (FACS) and Flow Cytometry Analysis of hENCCs

To enrich the hENCC population, the differentiated cells are subjected to fluorescence-activated cell sorting (FACS) at day 10. The cells are dissociated by Accutase for 15-20 min at 37° C. and then filtered through 40 μm cell strainer (Corning, 352340). The dissociated cells are then resuspended in DPBS with 2% (v/v) fetal bovine serum (FBS) (Thermo Fisher Scientific, 10270106) in a density of 1×10⁷ cells ml⁻¹ and stained with p75^{NTR}-FITC antibody (2 μl per 10⁷ cells; Miltenyi Biotec, 130-113-420) and HNK1-APC antibody (1 μl per 10⁷ cells; BD Biosciences, 560845) for at least 30 min on ice. The labelled cells are filtered through 35 mm cell strainer (Corning, 352235) before sorting. The percentage of p75^{NTR} and HNK1 double-positive cells is evaluated by flow cytometry and the double-positive cells were gated and sorted with BD FACSAria III Cell Sorter using purity mode with purity >99%. Unstained samples are used as the controls. FlowJo v10.7 (BD) is used to analyze flow cytometry data.

Neuronal Differentiation of hENCCs to ENS Neurons

The culture plate for neuronal differentiation is pre-coated with poly-L-ornithine (PO) (Sigma-Aldrich, P3655), laminin (Lam) (R&D Systems, 3400-010-02) and fibronectin (FN) (Thermo Fisher Scientific, 33016015) before cell seeding. In brief, each well of 24-well plate is coated with 15 μg ml⁻¹ PO in DPBS at 37° C. overnight. The PO-coated surface is rinsed with DPBS and then coated with 1 μg ml⁻¹ Lam and 10 μg ml⁻¹ FN in DPBS at 37° C. overnight. 5×10⁴ FACS-enriched hENCCs are resuspended as 5 μl droplets and seeded on pre-dried PO/Lam/FN-coated surface in N2 medium supplemented with 10 ng ml⁻¹ FGF-2, 10 μM Y-27632 and 3 μM CHIR99021. Neuronal differentiation of hENCCs to ENS neurons is initiated by replacing the medium with N2 medium supplemented with 10 ng ml⁻¹ BDNF, 10 ng ml⁻¹ GDNF, 10 ng ml⁻¹ NT-3 and 10 ng ml⁻¹ β-NGF, 1 μM dibutyryl-cAMP and 200 μM ascorbic acid. The cells are cultured in the neuronal differentiation medium up to 20 days and the medium was changed every 2-3 days.

Quantitative RT-PCR (qPCR)

Total RNA from HCOs and HBSOs are extracted by RNeasy Mini Kit (Qiagen) and the RNA concentrations are determined by Nanodrop 1000 Spectrophotometer (Thermo Fisher Scientific). The RNA is then reverse-transcribed to cDNA using HiScript III All-in-one RT SuperMix (Vazyme, R333-01). For real-time qPCR, cDNA samples are amplified by Luna Universal qPCR Master Mix (New England BioLabs, M3003) using specific primer pairs with PCR profiles of 95° C. (1 min) followed by 45 cycles of 95° C. (15 s) and 60° C. (30 s). Fluorescence is measured by ViiA 7 Real-Time PCR System (Thermo Fisher Scientific) at the end of each cycle. Each individual sample is assayed in triplicate and gene expression is normalized with GAPDH expression. The primers for qPCR analysis are listed in Table 1.

TABLE 1
List of RT-PCR primers used in the present invention

| | | | Product |
|---|---|---|---|
| | | Tm | size |
| Gene | Oligo sequence (5′ to 3′) | (° C.) | (bp) |
| HOXA2 | Forward: CGTCGCTCGCTGAGTGCCTG | 63.3 | 92 |
| | (SEQ ID NO: 03) | | |
| | Reverse: TGTCGAGTGTGAAAGCGTCGAGG | 61.1 | |
| | (SEQ ID NO: 04) | | |
| HOXB1 | Forward: GAGCTTTGCACCGGCCTAT | 58.1 | 103 |
| | (SEQ ID NO: 05) | | |
| | Reverse: CTTCATCCAGTCGAAGGTCCG | 57.4 | |
| | (SEQ ID NO: 06) | | |
| HOXB2 | Forward: CCTAGCCTACAGGGTTCTCTC | 56.1 | 79 |
| | (SEQ ID NO: 07) | | |
| | Reverse: CACAGAGCGTACTGGTGAAAAA | 55.4 | |
| | (SEQ ID NO: 08) | | |
| GBX2 | Forward: ACGTCAGCAGGTTCGCTATC | 57.2 | 166 |
| | (SEQ ID NO: 09) | | |
| | Reverse: AGCTGGGCTGTGACTTTGTT | 57.3 | |
| | (SEQ ID NO: 10) | | |
| OTX2 | Forward: AGTCGAGGGTGCAGGTATG | 56.6 | 153 |
| | (SEQ ID NO: 11) | | |
| | Reverse: GGCCACTTGTTCCACTCTCT | 57.1 | |
| | (SEQ ID NO: 12) | | |
| PAX7 | Forward: CAAACACAGCATCGACGGC | 57.3 | 91 |
| | (SEQ ID NO: 13) | | |
| | Reverse: CTTCAGTGGGAGGTCAGGTTC | 57.2 | |
| | (SEQ ID NO: 14) | | |
| OLIG3 | Forward: TCATGCTCACCAGCTCCCT | 58.7 | 58 |
| | (SEQ ID NO: 15) | | |
| | Reverse: CCCCCATAGATCTCGCCAAC | 57.6 | |
| | (SEQ ID NO: 16) | | |
| NGN1 | Forward: AAGACTTCACCTACCGCCC | 56.9 | 76 |
| | (SEQ ID NO: 17) | | |
| | Reverse: GGCGTTGTGTGGAGCAAGT | 58.6 | |
| | (SEQ ID NO: 18) | | |
| PHOX2B | Forward: AAACTCTTCACGGACCACGG | 57.4 | 222 |
| | (SEQ ID NO: 19) | | |
| | Reverse: CTCCTGCTTGCGAAACTTGG | 56.8 | |
| | (SEQ ID NO: 20) | | |
| GAPDH | Forward: CAAGAAGGTGGTGAAGCAGGC | 58.7 | 184 |
| | (SEQ ID NO: 21) | | |
| | Reverse: GCCAAATTCGTTGTCATACCAGGA | 57.6 | |
| | (SEQ ID NO: 22) | | |

### Western Blot

To extract proteins from HBSOs, HBSOs are lysed with Cell Lysis Buffer (1×) (Cell Signaling Technology, #9803) with PhosSTOP (Roche), Complete protease inhibitor cocktails (Roche) and 0.1 mM phenylmethylsulfonyl fluoride (PMSF) for 30 min on ice with vortex at intervals. The lysates are centrifuged at 10,000×g at 4° C. and the supernatant is collected. The protein extracts are separated by 12% SDS-PAGE and transferred to Immobilon-P PVDF membrane (Millipore, IPVH00010) using Trans-Blot® SD Semi-Dry Transfer Cell (Bio-Rad). The membrane is briefly washed with TBST and then blocked with blocking solution for 1 h at room temperature. After blocking, the membrane is incubated with 1:1000 rabbit anti-PHOX2B antibody (Abcam, ab) or 1:20,000 mouse anti-Actin antibody (Millipore, MAB1501) diluted in blocking solution at 4° C. overnight. The membrane is washed with TBST thrice for 10 min and then incubated with 1:2000 goat anti-rabbit immunoglobulins-HRP (Dako, P044801) or 1:5000 anti-mouse immunoglobulins-HRP (Dako, P044701) diluted in blocking solution for 1 h at room temperature. After washing thrice with TBST for 10 min, the HRP signals are detected using WesternBright ECL (Advansta, K-12045-D50).

### Immunofluorescence and Immunocytochemistry

The HCOs, HSBOs and embryonic mouse brains at E11.3, E13.5 and E15.5 are fixed with 4% (w/v) paraformaldehyde (PFA) in PBS at room temperature for 1-2 h or 4° C. overnight. After washing with PBS for 5 min thrice, the samples are dehydrated with 15% (w/v) sucrose in PBS and then with 30% (w/v) sucrose in PBS at 4° C. overnight. The dehydrated samples are embedded in Tissue-Tek O.C.T. Compound (Sakura, 4583) and frozen on dry ice. The frozen blocks are stored at −80° C. until sectioning. For immunofluorescence (IF), sections with 10-20 μm-thickness are cut using Leica CM3050 S cryostat and mounted on Superfrost Plus Adhesion Slides (Thermo Fisher Scientific, J1800AMNZ). The frozen sections are kept at −20° C. for storage. The frozen sections are warmed at 37° C. for 10-30 min before IF procedures and washed with PBS for 5 min twice with gentle shaking. The sections are blocked with 5% (v/v) donkey scrum in PBS with 0.1% (v/v) Triton X for 1 h at room temperature and then incubated with primary antibodies at 4° C. overnight. After washing with PBS for 10 min thrice, the sections are incubated with respective secondary antibodies for 2 h at room temperature. After washing with PBS for 10 min thrice, the stained cells are mounted with ProLong Diamond Antifade Mountant with DAPI (Thermo Fisher Scientific, P36971).

For immunocytochemistry (ICC), hENCCs and ENS neurons are fixed with 4% PFA in PBS for 20 min at room temperature. Fixed cells are blocked with 1% (w/v) bovine serum albumin (BSA) in PBS with 0.1% (v/v) Triton X for 1 h at room temperature and then incubated with corresponding primary antibodies at 4° C. overnight. After washing with PBS for 10 min thrice, the cells are incubated with respective secondary antibodies for 2 h at room temperature. After rinsing with PBS, the cells are stained with 1:2000 DAPI (Thermo Fisher Scientific, 62248) in PBS for 10 min at room temperature. After washing with PBS for 10 min twice, the stained cells are mounted with ProLong Diamond Antifade Mountant with DAPI. All the fluorescence images are acquired by Carl Zeiss LSM800 or LSM900 confocal microscope.

Droplet-Based Single-Cell RNA-Sequencing (scRNA-Seq)

HCOs are harvested at day 60 and digested with Accutase for 30 min at 37° C. with agitation. The HCOs are dissociated by pipetting every 10 min during digestion. All the dissociated cells are then filtered with 40 μm cell strainer and resuspended in 2% (v/v) FBS in DPBS in a density of 1×10⁷ cells ml⁻¹. The viable cells are labeled with 7-AAD (5 μl per 106 cells; BD Biosciences, 559925) for at least 10 min on ice. After filtering through 35 μm cell strainer, the cells negative for 7-AAD are gated and sorted with BD FACSAria III Cell Sorter using purity mode with purity ≥99%. The FACS-sorted cells are then subjected to droplet-based scRNA-seq using Chromium Single Cell platform and Single Cell 3′ Library Kits (10× Genomics) in Centre of PanorOmic Science (CPOS), The University of Hong Kong. In brief, cells are encapsulated into Gel Beads-in-emulsion (GEMs) by the 10× Chromium Single Cell Controller, followed by reverse transcription and library preparation to become a pool of cDNA libraries. Libraries are then purified and sequenced on an Illumina™ NextSeq 500 according to the manufacturer's protocol.

Pre-Processing of Droplet-Based scRNA-Seq Data

Cellranger toolkit (version 3.0) provided by 10× Genomics are used to perform the reads de-multiplexing and alignment. Unique molecular identifier (UMI) is counted by aligning to the human hg38 transcriptome. For the quality control, only cells with more than 800 detected genes are retained. Genes expressed in more than 10 cells are kept for further analysis. Clustering and marker identification are performed by the Seurat R package. The integrative analysis of the public datasets is also performed by Seurat.

### Dimensionality Reduction and Cell Clustering

The R package Seurat (version 3.1.4) implemented in R (version 3.6.2) are used to perform dimensional reduction analysis. The “NormalizeData” function from Seurat is used to normalize the raw counts, and the scale factor is set to 200,000, then followed by “FindVariableFeatures” with default parameters to calculate highly variable genes for each sample. After performing “JackStraw”, which returned the statistical significance of PCA (Principal component analysis) scores, 20 significant PCs are selected to conduct dimension reduction and cell clustering. Then, cells are projected in 2D space using t-SNE (1-distributed Stochastic Neighbor Embedding) with default parameters.

### Single Cell Data Integration and Batch Effect Correction

To account for batch effect among different samples, “FindIntegrationAnchors” is used in the Seurat package to remove batch effect and merge samples to one object. In detail, the top 4,500 genes with the highest expression and dispersion from each sample are used to find the integration anchors, and then the computed anchoret is applied to perform dataset integration.

### Organoid Comparison

Human brainstem organoids (HBSOs) and human cerebral organoids (HCOs) are derived from the sequencing data in the present invention, while human cortical organoids (hFBCOs, GSE137941), human midbrain organoids (hMBOs, GSE133894), human thalamic organoids (hThaOs, GSE122342) and human arcuate organoids (hARCOs, GSE164102) are downloaded from Gene Expression Omnibus (GEO) database. Raw count matric of UMI are downloaded and normalized by NormalizeData function in Seurat R package. Cells from each organoid physical models are pooled together to construct “pseudo-bulk” expression for each organoid. Similarities between different organoids are estimated by Spearman correlation.

### Reference-Based Cell Annotation

Developing and adolescent neuron, glia, OPC, radial glia, Schwann cell and astrocyte located at hindbrain are extracted from developing and adolescent mouse brain atlas for comparison with the HBSO scRNA-seq dataset. Reference-based cell annotation is performed by Seurat R package. In brief, raw count matric are normalized and scaled first. Then, FindTransferAnchors function is applied to find shared anchors between datasets. Finally, MapQuery is used to project query dataset (HBSOs) to reference datasets (developing and adolescent mouse brain cells). Thus, original cell type labels are transferred to HBSOs datasets based on the overall expression patterns of genes and cells in HBSOs are classified to six main cell types (neuron, glia, OPC, radial glia, Schwann cell and astrocyte).

Human cerebral organoids from public scRNA-seq datasets are downloaded from EMBL's European Bioinformatics Institute. 2-month-old human cerebral organoids reextracted and overlaid with our HCO cells for comparison analysis. Potential technical and batch effects are removed by Seurat software. The sixteen unbiased clusters are further classified into six cell types, including neural stem cells (NSC), radial glia cells (RGC), intermediate progenitors (IP), cortical neurons (CN), midbrain/hindbrain (M/H) and mesenchymal-like cells (MC) based on the expression of canonical markers and the cell annotation from the public datasets.

### Marker-Based Neuronal Subtype Annotation

Neuronal cells in HBSOs are further classified into four main neuron subtypes (Cholinergic, Serotonergic, GABAergic and Glutamatergic) and two novel subtypes (PHOX2B^{Low} and PHOX2B^{High}) based on the canonical marker genes as stated in previous literature. Gene signature expression of each subtype is calculated to confirm the identity of neuronal cells.

### Lineage Cladogram Construction

Pseudo-bulk expression (average expression) of each cell type is calculated. Pairwise Euclidean distances are calculated among cell types. Neighbor-joining (NJ) tree is estimated based on the distance matrix ggtree. R package is used to visualized the lineage cladogram.

### Single Cell Trajectory Analysis

Single cell trajectory analysis is performed by PCA algorithm using PHOX2B⁺ cells.

### Identification of Differentially Expressed Genes and Pathway Enrichment Analysis

To identify unique differentially expressed genes (DEGs) among each cluster, the “FindAllMarkers” function from Seurat is used and non-parametric Wilcoxon rank sum tests are set to evaluate the significance of each individual DEG. The DEG analysis between mutant and control based on single-cell expression data is performed using monocle (version 2.14.0) R package. The DEGs with adjusted P-value less than 0.05 and Log₂ Fold Change (log 2FC) larger than 0.5 are thought to be significant and used in downstream analysis. Gene ontology (GO) term enrichment analyses are performed using clusterProfiler (version 3.14.3) R package. Terms that had an adjusted P-value <0.05 is defined as significantly enriched.

### Pathway Score Analysis

In order to measure the activity of whole pathways between samples at transcriptome level, a simple additive model that ignores the interactions between genes is applied to estimate the overall expression level of the pathways. The genes involved in GO:0007389 (pattern specification process) in GO database are used to evaluate the overall changes of the key biological processes.

### Gene Regulatory Network (GRN) Analysis

Single cell regulatory network inference and clustering (SCENIC, version 1.1.2) is used to infer transcription factor networks active using scRNA-Seq data. Analysis is performed using default and recommended parameters as directed on the SCENIC vignette using the hg38 RcisTarget database. Kernel density line histograms showing differential AUC score distribution across conditions are plotted with ggplot2 v.3.1.1 using the regulon activity matrix (‘3.4_regulonAUC.Rds’, an output of the SCENIC workflow) in which columns represent cells and rows the AUC regulon activity. Fold-change (FC) difference between median AUC values is calculated and the highest changed TFs are plotted.

### Differential Co-Expression Analysis

To predict PHOX2B activated and repressed target genes, a three-step strategy is applied to obtain the target genes of PHOX2B during progenitor-to-neuron transition. In the first step, the significantly correlated genes to PHOX2B which represented the genes that had strong regulatory relationships with PHOX2B are identified. In the second step, the differential expression profile between control and mutant is utilized to filter out the genes whose expression levels are not significantly disrupted by PHOX2B. Finally, to confine the genes to be the actual targets of PHOX2B, a public ChIP-seq dataset designed for PHOX2B from a similar cell state (neuronal progenitor) in Cistrome database is utilized to obtain the direct/indirect binding genes of PHOX2B. Activated and repressed target genes are determined by the positive/negative correlation and up/down-regulation of the differential expression, respectively. In addition, TF motif enrichment analysis by FIMO tool of MEME suite is used to cross-validate the predicted activated targets of PHOX2B.

### Imputation

Markov affinity-based graph imputation of cells (MAGIC) is used to denoise the cell count matrix and fill in missing transcripts. The expression of PHOX2B can be well-recovered and most of the PHOX2B+ cells are found in cell clusters inferred to be less mature progenitors (IP) or more mature neurons (CN), and few fell into NSC in control HCOs.

### TF Correlation Network

The TF co-expression network is constructed by WGCNA according to the correlation of the genes. Nodes (TFs) with more than three edges and edges with a high correlation (>0.2) are kept in the network. Cytoscape is used to visualize the TF correlation network.

### Example 1. Generation and Characterization of Human Brainstem Organoids (HBSOs) from hPSCs

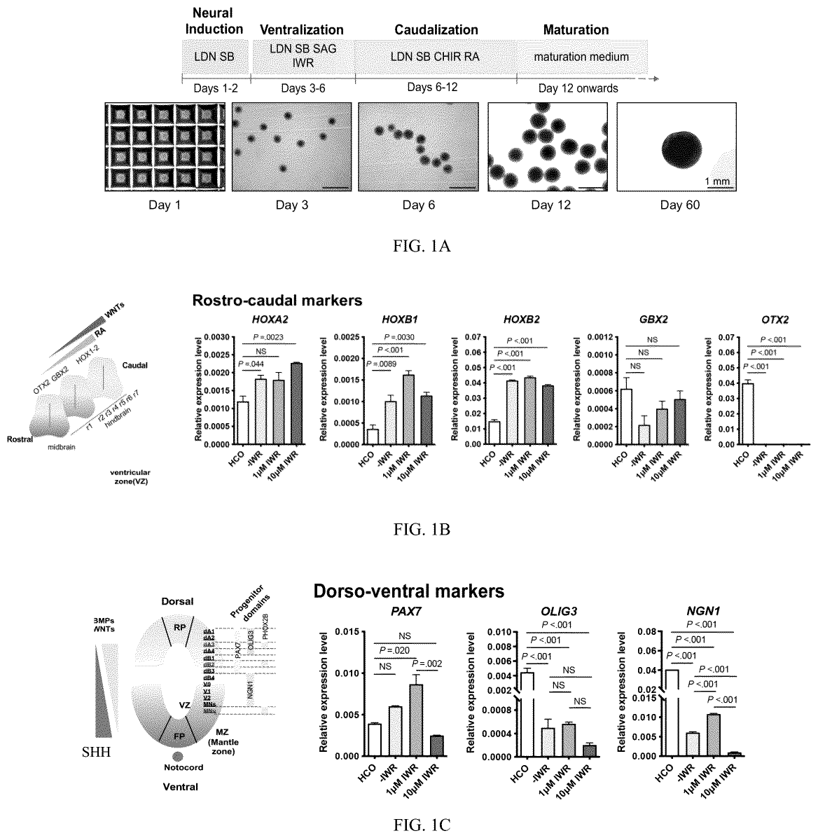

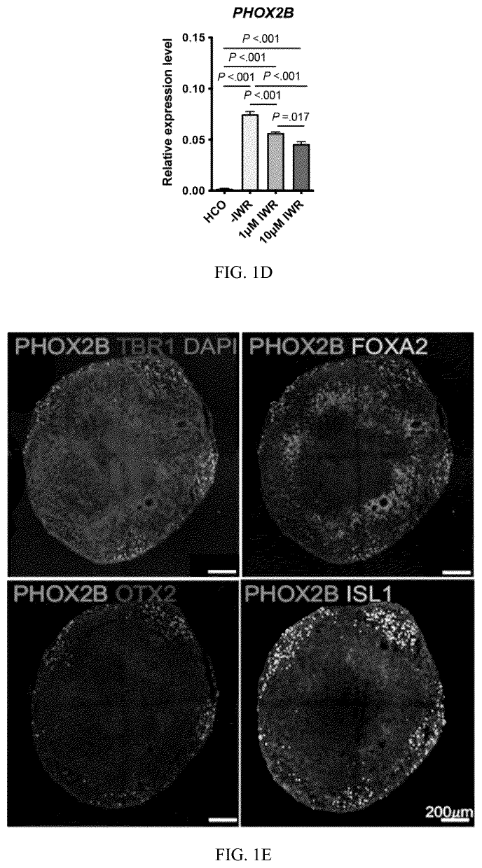

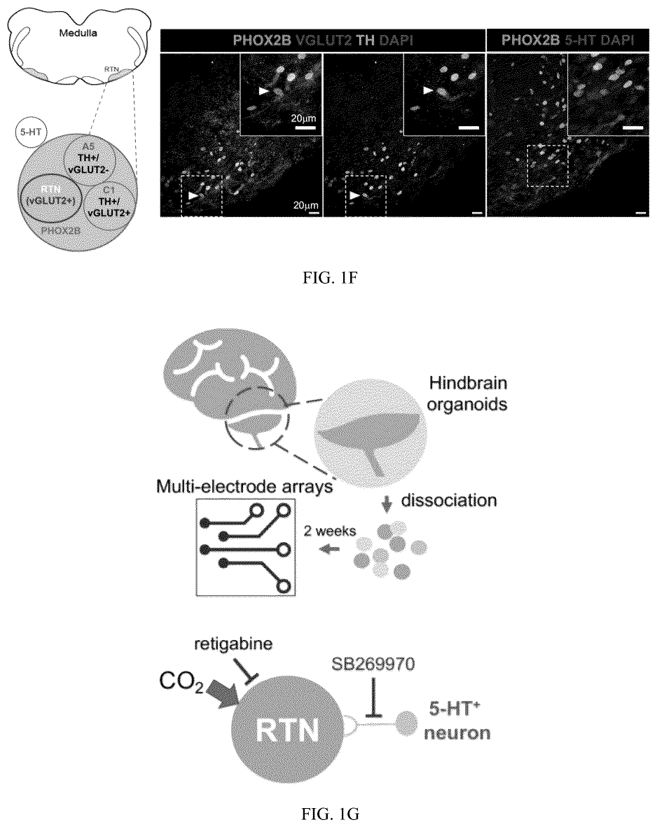

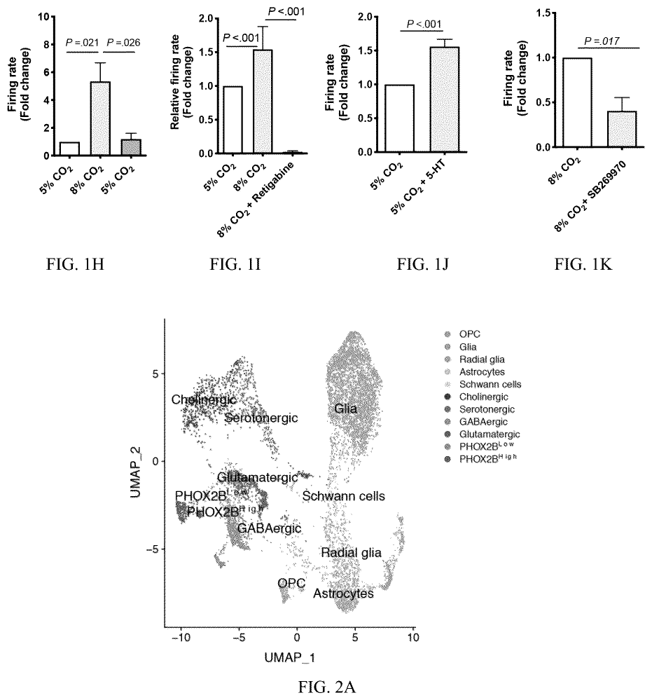

To model the CCHS condition, a protocol for generating human brainstem organoids (HBSOs) that include the PHOX2B⁺domains and RTN neurons has been developed. A control human pluripotent stem cell (hPSC) line (UE02306, provided by Dr. Guangjin Pan, Guangzhou Institutes of Biomedicine and Health, Chinese Academy of Sciences, China) is used to test the differentiation protocol. Initially, the hPSCs are directed to the neural cell fate using dual-SMAD inhibitors (LDN193189 and SB431542), followed by ventralization with a SHH agonist (SAG), and subsequent caudalization to the hindbrain cell fate with a WNT agonist (CHIR99021) and retinoic acid (RA) (FIG. 1A). RTN neurons originate from rhombomere 5 (r5) and the dB2 domain during hindbrain development, which requires moderate WNT signaling levels (FIGS. 1B and 1C). Therefore, WNT signaling is fine-tuned by adjusting the concentrations of the WNT antagonist IWR1-endo; three different concentrations (0, 1, or 10 μM) of IWR1-endo are added to the culture during dorso-ventral patterning on days 3-6. By day 20 of differentiation, HBSOs are harvested and subjected to RT-qPCR to examine the expressions of various rostro-caudal and dorso-ventral markers.

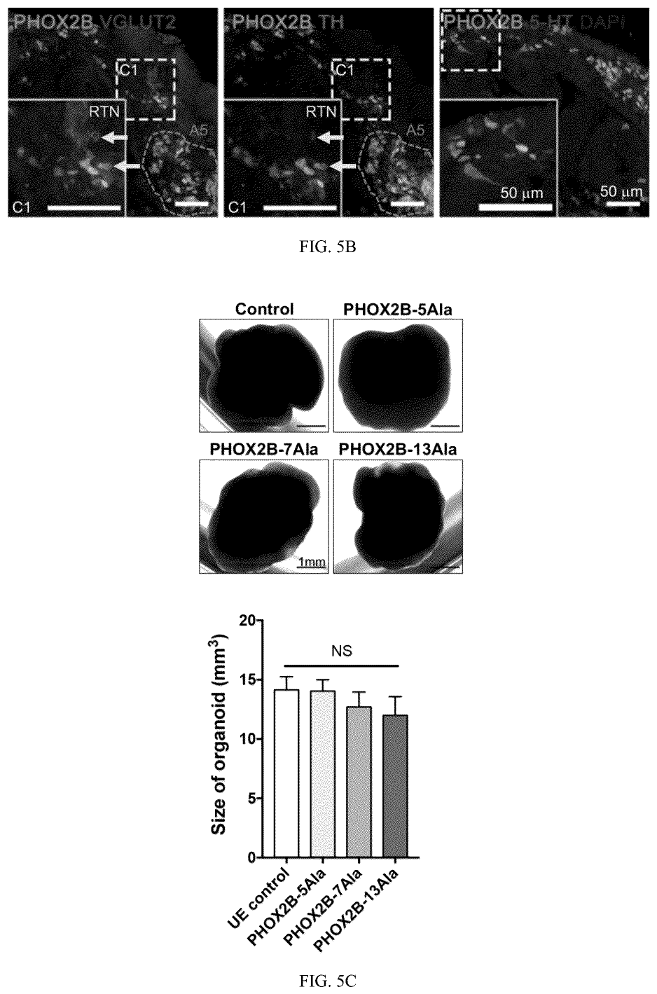

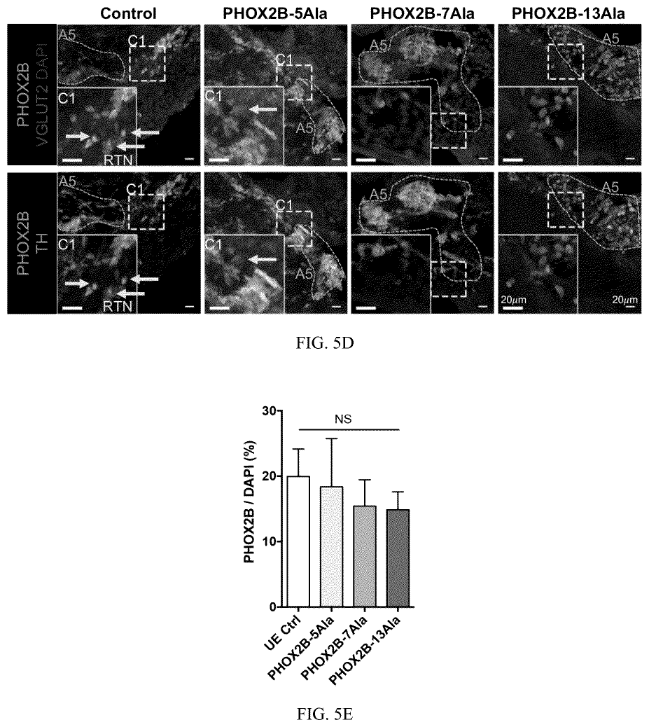

Compared to human cerebral organoids (HCOs) derived using an unguided differentiation protocol and containing various discrete brain regions, HBSOs expressed significantly higher levels of r4-5 markers (HOXA2, HOXB1 and HOXB2), a lower level of the r1 marker (GBX2), and undetectable levels of the midbrain marker (OTX2) (FIG. 1B). HBSOs cultured without or with higher concentrations of IWR1-endo (0 or 10 μM) express slightly lower levels of the r4 marker (HOXB1), indicating specification to the r5 domain. Regarding dorsal-ventral specification, these HBSOs express lower levels of PAX7, OLIG3, and NGN1 compared to HCOs (FIG. 5C). Consistent with their spatial identity highly resembling the dB2 domain of the neural tube, a much higher expression level of PHOX2B is observed in HBSOs, with the highest expression in those cultured without IWR1-endo (FIG. 1D). Lowering WNT levels by adding IWR1-endo (1 μM or 10 μM) does not provide additional benefits in favoring the dB2 domain formation; moreover, the addition of a high concentration of IWR1-endo (10 μM) inhibited the growth of the HBSOs. Therefore, the HBSOs generated without IWR1-endo are cultured for another 40 days in maturation medium.

By day 60, the HBSOs include many PHOX2B⁺ cells in the outer layer, exhibiting a hindbrain signature as revealed by immunostaining with region-specific markers. Most PHOX2B⁺ cells co-express the hindbrain marker ISL1 but not the dorsal midbrain marker OTX2 nor the forebrain marker TBR1 (FIG. 1E). Despite the presence of some ventral midbrain FOXA2⁺ neurons in the middle layer of the HBSOs, no PHOX2B signal is detected in this neuron layer, suggesting that PHOX2B⁺ neurons likely retain the specific position codes for hindbrain neurons. In the respiratory center of the brainstem, PHOX2B-expressing neurons are found in three domains: glutamatergic neurons (PHOX2B⁺; VGLUT2⁺; TH⁻) in the RTN, tyrosine hydroxylase (TH)-expressing neurons (PHOX2B⁺; TH⁺; VGLUT2⁻) in the A5 domain, and PHOX2B⁺; TH⁺; VGLUT2⁺ neurons in the Cl domain. These PHOX2B-expressing domains are adjacent to serotonin (5-HT)-expressing neurons, as shown in FIG. 1F (left panel). In the day 60 HBSOs, most of these PHOX2B⁺domains are identified, with PHOX2B⁺ glutamatergic (PHOX2B⁺; VGLUT2⁺; TH⁻) RTN-like neurons residing close to tyrosine hydroxylase (TH)-expressing A5 (PHOX2B⁺; TH⁺; VGLUT2⁻) and Cl (PHOX2B⁺; TH⁺; VGLUT2⁺) neurons, as well as next to the major afferent inputs of RTN neurons, the serotonin-expressing (5-HT+) neurons (FIG. 1F, right panel).

The functionality of HBSO neurons, particularly their responses to hypercapnia, is further assessed using multi-electrode array (MEA) analyses. Briefly, Day 60 HBSOs arc dissociated with Papain Dissociation System (Worthington, LK003150) following the manufacturer's protocol. In brief, the HBSOs are digested with the papain solution in 37° C. incubator for 30 min with continuous shaking at 90 r.p.m. and then triturated with P1000 pipette. The triturated HBSOs are further digested in 37° C. incubator for an additional 10 min and then filtered through 40 μm cell strainer (Corning, 352340) to remove undigested cell debris. The dissociated cells are collected by centrifuging at 300×g for 5 min at room temperature. The cells are then resuspended with DNase/ovomucoid inhibitor solution to stop the digestion. The cells are centrifuged at 300×g for 5 min at room temperature and resuspended in BrainPhys Neuronal Medium (STEMCELL Technologies, #05790) supplemented with 0.5% N-2 supplement (v/v), 1% B-27 supplement (v/v), 1% GlutaMAX (v/v), 0.5% NEAA, 2.5 μg ml⁻¹ insulin, 20 ng ml⁻¹ BDNF, 20 ng ml⁻¹ GDNF, 50 nM dibutyryl-cAMP, 200 μM ascorbic acid, 49.5 M β-mercaptoethanol and 1% (v/v) Pen-Strep. Five-microliter cell suspension with 1 × 105 cells and 10 μg ml⁻¹ laminin is seeded into each well of 24-well CytoView MEA plate (Axion Biosystems, M384-tMEA-24W) pre-coated with 0.1% polyethylenimine (PEI) (Sigma-Aldrich, P3143) and maintained in 37° C. humidified 5% CO₂ incubator for at least 2 weeks before MEA analysis. The culture medium is changed every 3-4 days. MEA recordings are measured at 37° C. with 5-8% CO₂ in a Maestro Edge MEA System (Axion Biosystems) to detect spontaneous firing of spikes. The MEA plate is pre-equilibrated in the MEA system for at least 5 min before measurement and each treatment is recorded for 5-10 min to calculate the mean spike firing rate. For the pharmacological experiment, retigabine (Selleckchem, S4733) (10 μM), 5-HT (Abcam, ab 120528) (10 μM) and 5-HT receptor blocker (SB269970) (Abcam, ab 120508) (10 μM) is applied to plate immediately before recording. The MEA recordings are analyzed using the Neural Module of AxIS Navigator 1.5 software and the Neural Metric Tool software (Axion Biosystems). The mean spike firing rate of each MEA recording is normalized with the first MEA recording undertaken in 5% CO₂ or 8% CO₂ condition to calculate the fold change.

HBSOs are first dissociated and re-seeded onto the MEA plate for 1-2 weeks to allow for the re-establishment of neuronal connectivity among cells and with the electrodes on the plate. Neural activities are then measured under different CO₂ levels (5% or 8% CO₂), in the presence of an RTN neuron blocker (KCNQ channel activator: 10 μM retigabine) or serotonin (10 μM 5-HT) and its receptor blocker (10 μM SB269970) (FIG. 1G). Consistently, hypercapnia-responsive neurons that exhibit increased firing in response to elevated CO₂ levels are detected (FIG. 1H). This elevated firing is RTN-dependent and can be blocked by the RTN-specific blocker retigabine (FIG. 1I).

Additionally, 5-HT is capable of enhancing the firing of HBSO neurons (FIG. 1J), and their hypercapnia responses are likely dependent on serotonin inputs, as evidenced by the counteraction of these responses by the 5-HT antagonist SB269970 (FIG. 1K). These findings suggest potential interconnections between 5-HT inputs and RTN neurons, similar to those found in the respiratory center of the mouse brain. In summary, the HBSOs contain hypercapnia-responsive neurons organized as a functional unit resembling the RTN-respiratory center.

### Example 2. Cell Type Diversity and Hindbrain Molecular Signatures of HBSOs

To further investigate the cellular composition and molecular signatures of HBSOs, single-cell transcriptomic analyses of HBSOs and HCOs derived from the control hPSC (UE02306) line at day 60 of differentiation are performed.

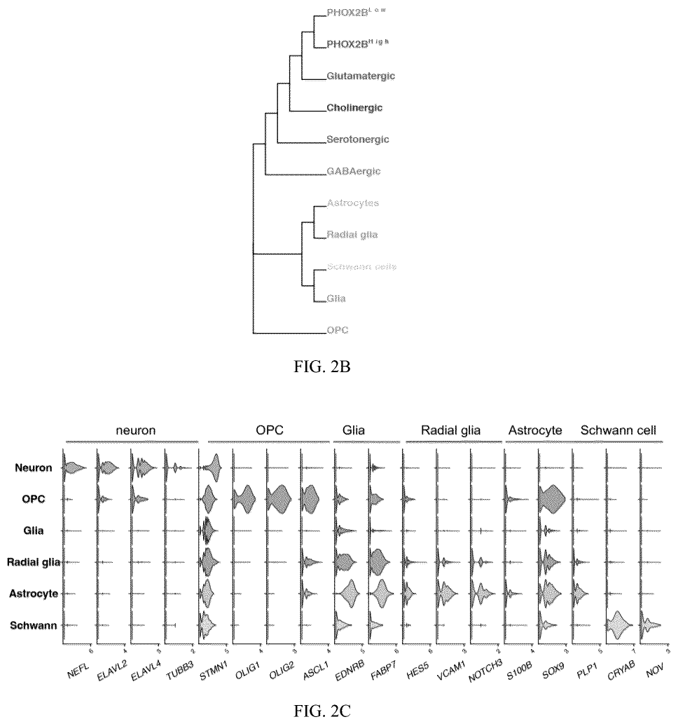

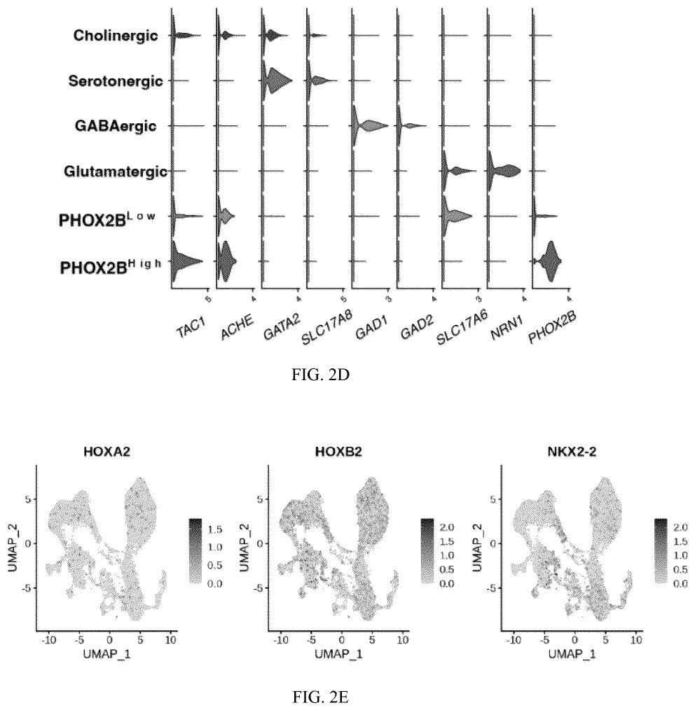

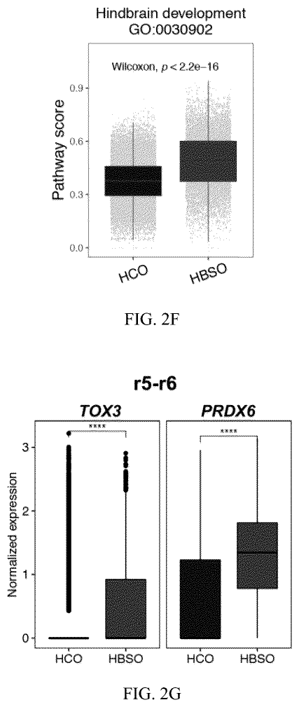

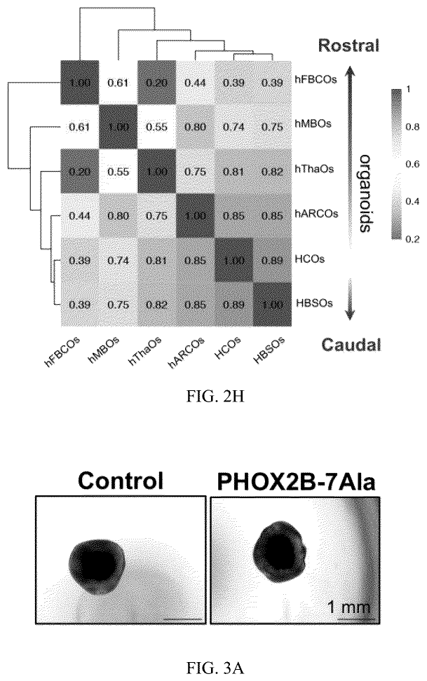

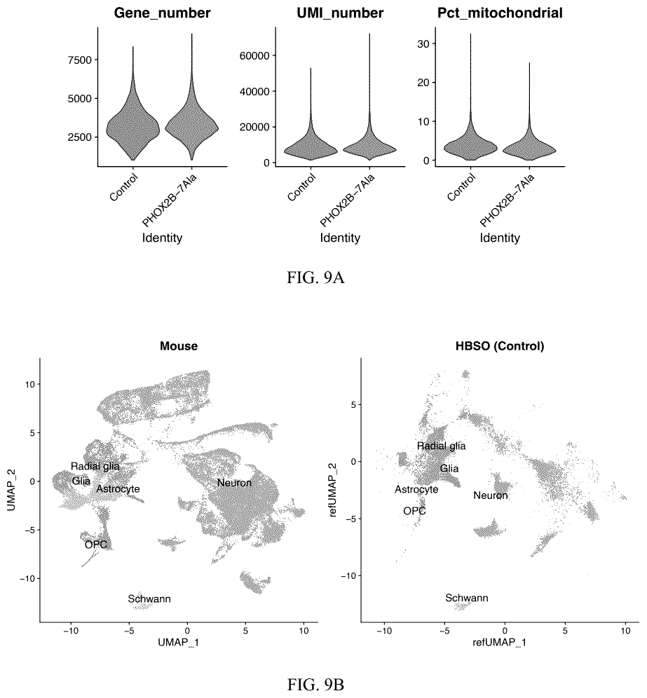

A total of 11,347 and 42,970 cells from a pool of 4 HBSOs and 8 HCOs, respectively, are sequenced using the Chromium system (10× Genomics), with 2,319 median genes and 24,389 mean unique molecular identifiers (UMIs) per cell (FIG. 9A). All the single cells are projected on Uniform Manifold Approximation and Projection (UMAP) plots. A reference-based cell annotation approach is conducted to identify the cell types in HBSOs. Single-cell RNA-sequencing (scRNA-seq) datasets of hindbrain cells from developing (embryonic day (E) 7-E18) and adolescent mouse brains are used to compare to the HBSO cells. Eleven main cell types, including six subclasses of neurons (cholinergic, serotonergic, GABAergic, glutamatergic, and two groups of PHOX2B⁺ neurons), oligodendrocyte progenitor cells (OPCs), glia, radial glia, astrocytes, and Schwann cells, are identified in HBSOs (FIG. 2A). Lineage relationships of these cell types are further demonstrated in the cladogram, where two distinct lineages for neuronal and glial cells are observed (FIG. 2B). The six subclasses of neuronal cells account for approximately 40% of the total HBSO cells, and they all express pan-neuronal markers (NEFL, ELAVL2, ELAVL4, and TUBB3), while 45% of the HBSO cells express glial markers (EDNRB and FABP7). Other cells exhibit molecular signatures resembling radial glia, astrocytes, Schwann cells, and a small population of OPC-like cells expressing OLIG1, OLIG2, and ASCL1 (FIG. 2C).

The four subclasses of neurons: cholinergic (TAC1⁺, ACHE⁺), serotonergic (GATA2⁺, SLC17A8⁺), GABAergic (GAD1⁺, GAD2⁺) and glutamatergic (SLC17A6⁺, NRN1⁺), are classified using unsupervised clustering analysis, followed by a marker-based cell annotation approach. The expression of canonical marker genes for these neuronal subtypes is shown in FIG. 2D. Additionally, there are two distinct groups of PHOX2B-expressing neurons with differential expression levels of PHOX2B (PHOX2B^{Low} and PHOX2B^{High}) (FIG. 2D). PHOX2B^{Low} cells also express the glutamatergic marker (SLC17A6, which encodes VGLUT2), representing the RTN-like neurons, while PHOX2B^{High} neurons are the cholinergic population of the PHOX2B⁺domain.

Similar to day 20 organoids, HBSOs at day 60 consistently express the caudal hindbrain markers (HOXA2, HOXB2, and NKX2-2). Analysis of the global gene expression profiles reveals that pathways associated with hindbrain development (GO:0030902) are highly enriched in HBSOs compared to HCOs. In particular, the expression of region-specific markers for r5 (TOX3) and r6 (PRDX6) is detected in HBSO cells. HBSOs are then compared with human whole brain cerebral organoids (HCOs) and other subregion organoids, including human forebrain organoids (hFBCOs, GSE137941), human midbrain organoids (hMBOs, GSE133894), human thalamic organoids (hThaOs, GSE122342), and human arcuate organoids (hARCOs, GSE164102) derived from hPSCs, representing the rostral-to-caudal regions of the brain. HBSOs consistently show the highest correlation with HCOs, and their correlations to the rostral subregion organoids (hFBCOs and hMBOs) are significantly lower when compared to hThaOs and hARCOs. In sum, HBSOs include all the major neural cell types found in the mouse hindbrain/brainstem, including glutamatergic PHOX2B⁺ neurons, and exhibit unique hindbrain-specific molecular signatures, making them distinctive from other subregion organoids.

### Example 3. PHOX2B-7Ala Prohibits the Generation of Hindbrain/RTN Neurons as Revealed by HBSO Physical Model

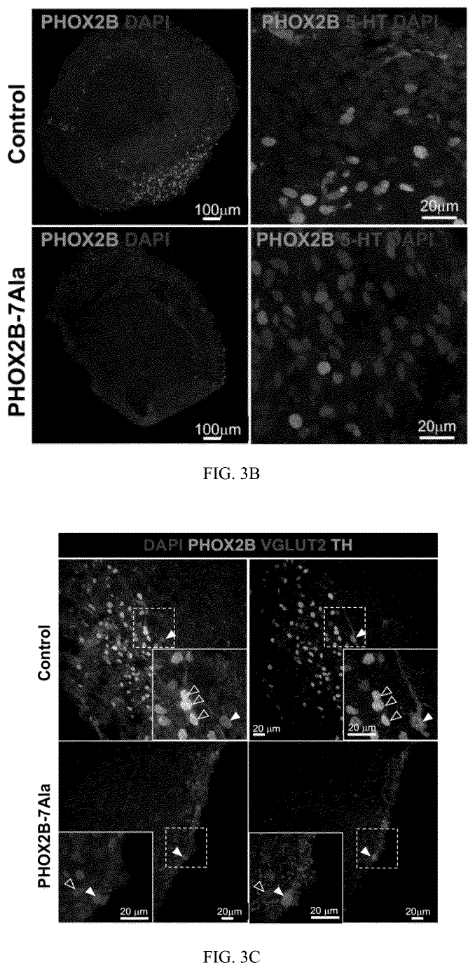

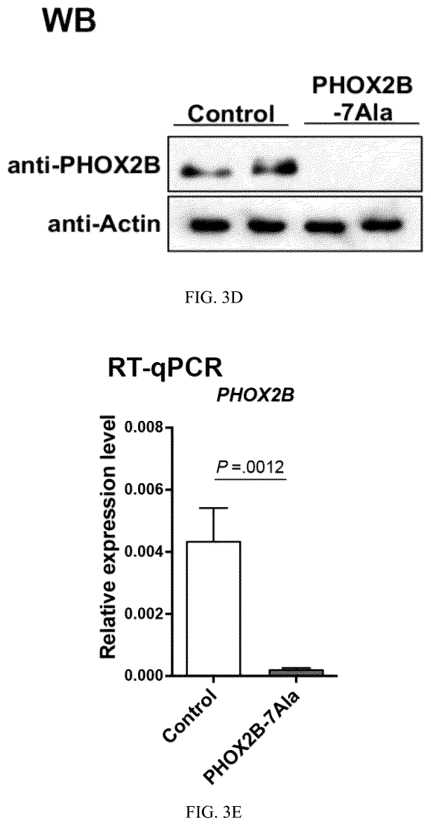

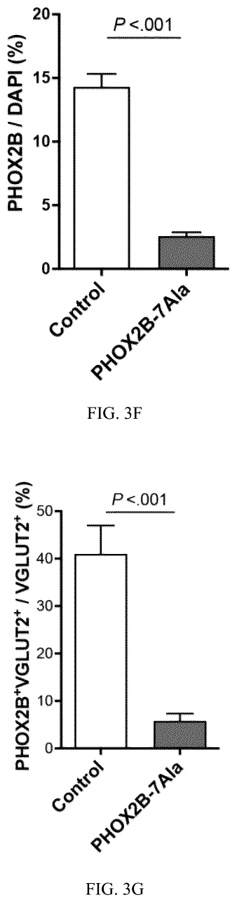

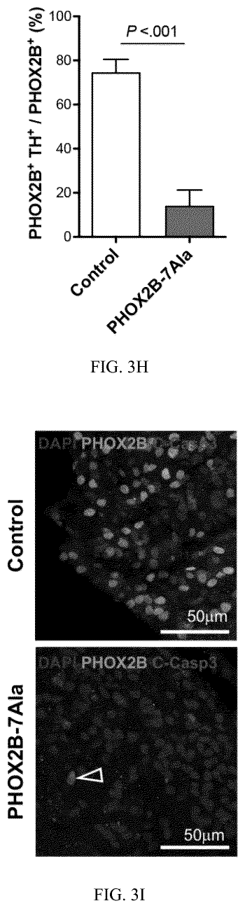

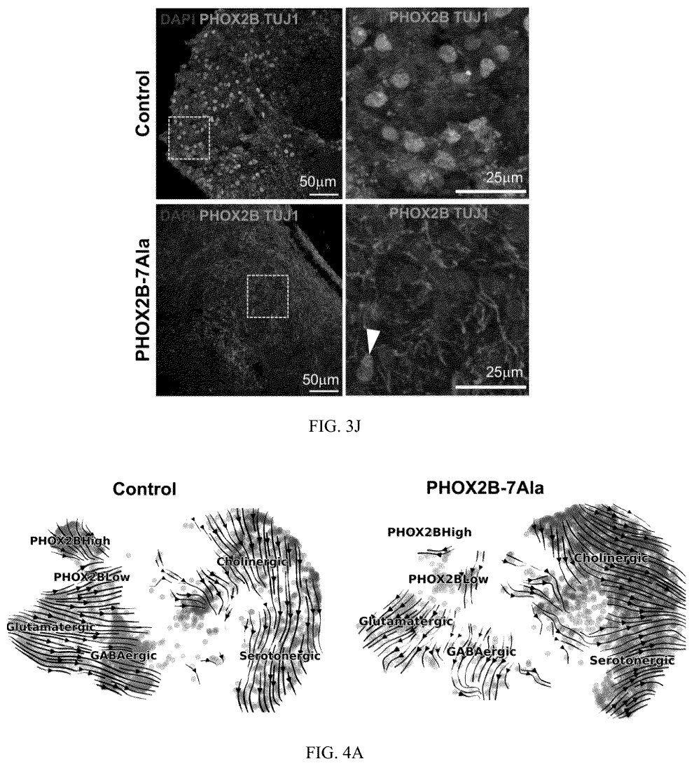

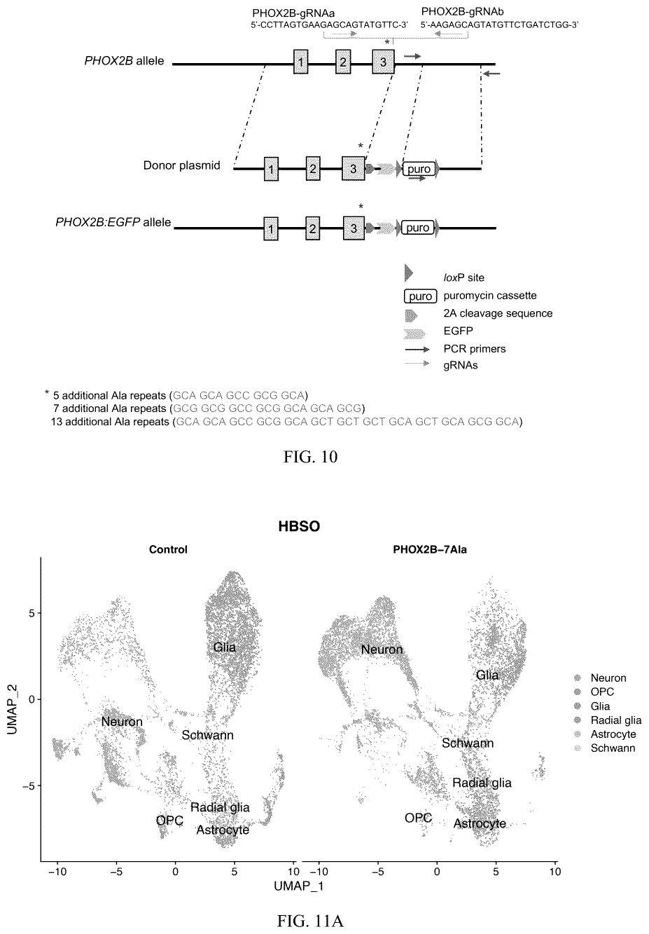

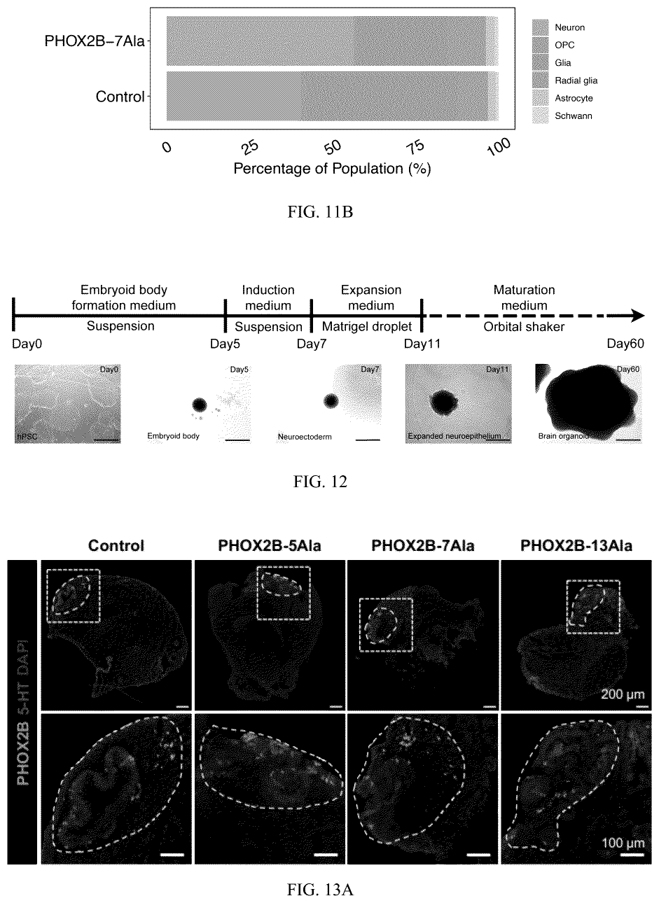

The HBSOs is used to examine how PHOX2B-PARMs affect the development of RTN neurons. PHOX2B-7Ala which carries an extra seven alanine repetition is the most common form of PHOX2B-PARMs found in CCHS-HSCR patients. Thus, this mutation is evaluated for generating an isogenic mutant hPSC line (PHOX2B-7Ala) from the control hPSC (UE02306) line. Using CRISPR-Cas9-mediated homology-directed repair platform with two guide-RNAs (gRNAs) specifically targeting exon 3 of PHOX2B locus, seven additional trinucleotide repeats are introduced in the polyalanine tract (+7Ala) of PHOX2B locus (FIG. 10). An in-frame insertion of an enhanced green fluorescent protein (EGFP) with a 2A self-cleaving peptide is added right after the stop codon of the PHOX2B to allow the subsequent detection of cells expressing the PHOX2B mutant protein (FIG. 10). Heterozygous +7Ala PHOX2B is confirmed by Sanger sequencing. PHOX2B-7Ala-hPSC are then used to generate HBSOs using the newly established HBSO differentiation protocol. The control and PHOX2B-7Ala HBSOs are highly comparable in size at day 60 (FIG. 3A). However, very few PHOX2B⁺ cells are found in the PHOX2B-7Ala HBSOs, and they were mainly located in the outer layer of HBSOs and in adjacent to 5-HT⁺ neurons (FIG. 3B). The generation of PHOX2B⁺TH⁺ neurons and PHOX2B⁺VGLUT2⁺TH⁻ RTN neurons are severely disrupted since the expression of PHOX2B is almost completely lost in the PHOX2B-7Ala HBSOs (FIG. 3C). The loss of PHOX2B-expressing cells in the day 60 PHOX2B-7Ala HBSOs is further confirmed by Western blot (FIG. 3D), RT-qPCR (FIG. 3E) and immunostaining (FIG. 3F) analyses. The only few PHOX2B⁺ neurons found in the mutant HBSOs only exhibit very low expression level of PHOX2B, as a result, the percentages of PHOX2B⁺VGLUT2⁺ (FIG. 3G) and PHOX2B⁺TH⁺ (FIG. 3H) neurons are also drastically decreased in the PHOX2B-7Ala HBSOs. In the day 60 control and PHOX2B-7Ala HBSOs, all PHOX2B⁺ neurons are negative for apoptotic marker (cleaved-Caspase3, C-Casp3⁻) (FIG. 3I) and express the pan-neuronal marker (TUJ1) (FIG. 3J), suggesting that the loss of PHOX2B⁺ neurons in mutant HBSOs is unlikely due to the detrimental effect of 7Ala-PARM on the cell survival or neuronal differentiation.

### Example 4. Characteristics of PHOX2B-7Ala Derived HBSOs Exhibiting Aberrant Neuronal Differentiation Paths and Hindbrain Signatures

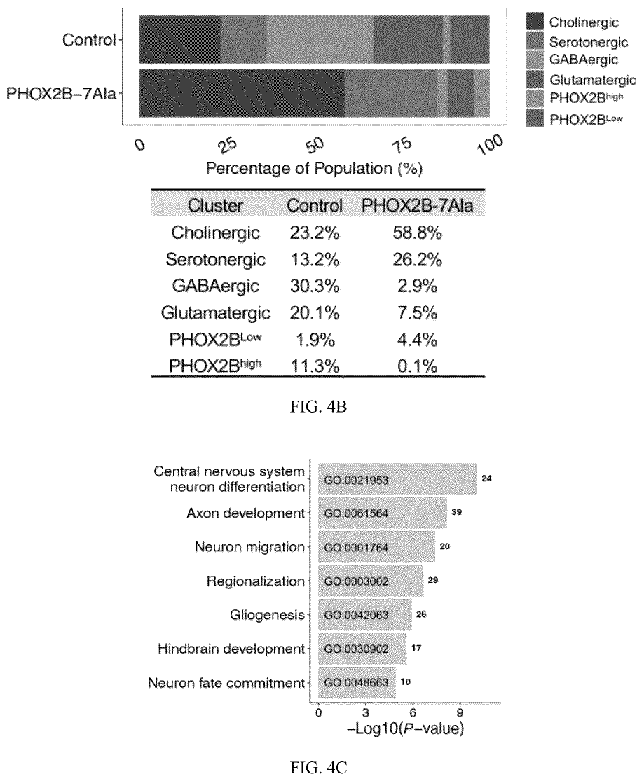

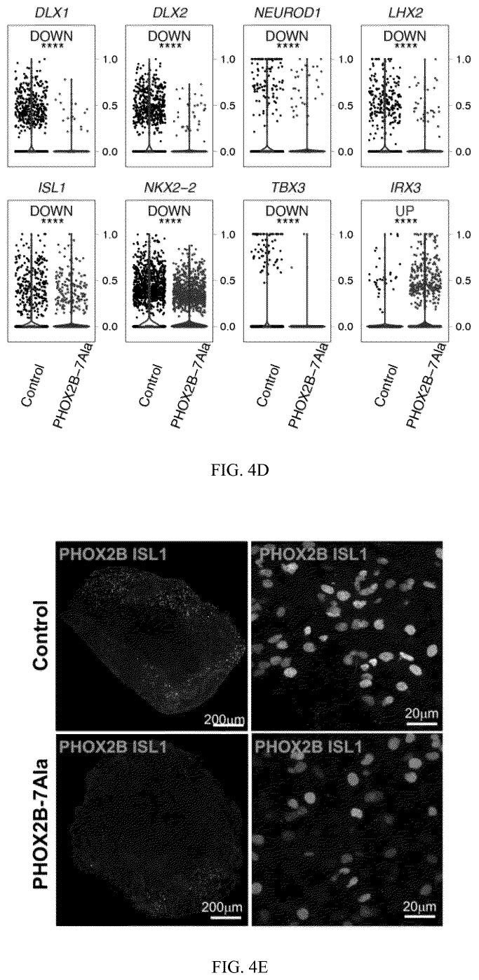

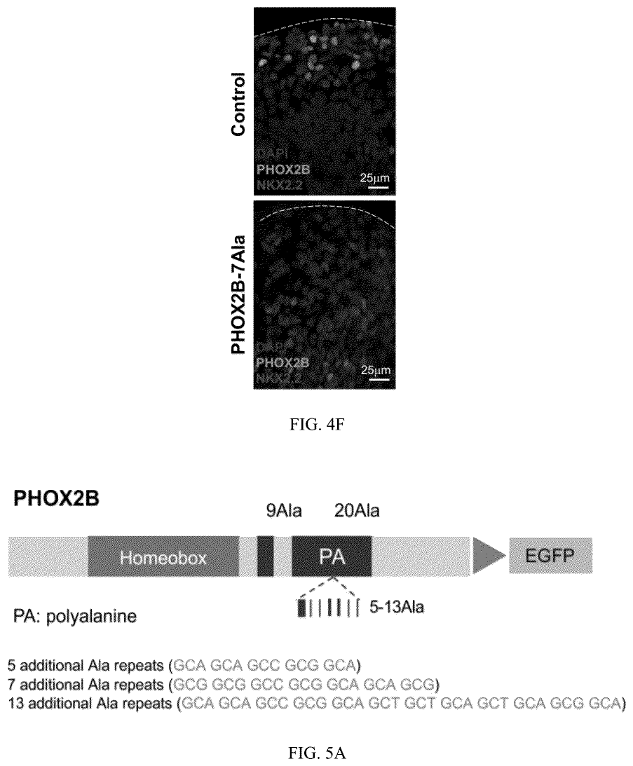

Currently, it is still unclear how PHOX2B-7Ala interferes the formation of respiratory unit. To delineate the underlying mechanisms, additional 11,741 cells from PHOX2B-7Ala HBSOs are sequenced using 10×Genomics. Despite that the main subtypes of neuronal and glial cells can be identified in PHOX2B-7Ala HBSOs, an obvious change in cellular composition of neuronal population is observed (FIGS. 11A-11B). RNA velocity analysis is conducted to evaluate the neuronal differentiation paths in PHOX2B-7Ala HBSO cells. Velocyto is used to calculate spliced and unspliced matric from bam files that derived from cellranger pipeline. Then, scVelo is applied for RNA velocity analysis in single cells. The results reveal that the neuronal differentiation paths are disrupted in PHOX2B-7Ala HBSO cells (FIG. 4A). The differentiation paths for both glutamatergic and GABAergic lineages are interrupted, and that are accompanied by the enlarged proportions of cholinergic and serotonergic like cells in PHOX2B-7Ala HBSOs. These alternations are associated with reduced PHOX2B expression, the PHOX2B^{High} population is almost completely diminished and most of PHOX2B expressing cells only expressed minimal level of PHOX2B in PHOX2B-7Ala HBSOs (FIGS. 4A and 4B). These observations suggest the cell-autonomous and non-cell autonomous effects of PHOX2B-7Ala in the development of respiratory unit. By comparing the transcriptomes of the neuronal cells in control and PHOX2B-7Ala HBSOs at single cell level, 642 differentially expressed genes (DEGs) are identified. With pathway analysis, these DEGs are enriched for “neuron differentiation/migration/fate commitment”, “regionalization” and hindbrain development (FIG. 4C). As shown in FIG. 4D, genes associated with neuronal differentiation, such as DLX1, DLX2, NEUROD1 and LHX2, are consistently down-regulated in PHOX2B-7Ala neurons, and genes implicated in hindbrain morphogenesis (ISL1, NKX2-2 and TBX3) and the development of midbrain-hindbrain boundary (MHB) (IRX3) are also dysregulated. Subsequent immunostaining of the hindbrain marker, ISL1 and PHOX2B, demonstrates that most of the PHOX2B⁺ cells do not co-express ISL1, suggesting the potential loss of hindbrain identity of these cells and/or with patterning defect (FIG. 4E). In line with this observation, the NKX2.2⁺ domain (the inferred mantle layer of the hindbrain) shifted from the outer layer in control HBSOs to the middle layer in the PHOX2B-7Ala HBSOs (FIG. 4F), further indicating that PHOX2B-7Ala perturbs the patterning of HBSOs.

### Example 5. Depletion of PHOX2B⁺ Glutamatergic Neurons in PHOX2B-PARM HCOs

The non-cell autonomous effect of PHOX2B-7Ala in hindbrain patterning is first reported in the mouse mutants expressing Phox2b-7Ala, where the Phox2b expressing glutamatergic (Phox2b⁺vGlut2⁺) neurons in RTN is almost abrogated, while they are mislocated and clustered dorsally to nVII. Therefore, how PHOX2B-PARMs perturb the development of respiratory unit, particularly the hindbrain patterning, is further evaluated. To this end, two additional PHOX2B mutant hPSC lines, carrying +5Ala and +13Ala in PHOX2B (FIG. 5A), are generated. In addition, an unguided differentiation protocol is employed to generate human HCOs with regional diversity as previously described, where subregions of the brain are self-organized, allowing the impacts of PARMs on brain patterning to be addressed. In brief, hPSCs are seeded in embryoid body formation medium and cultured for 5 days to allow the generation of 3-D embryoid bodies which will then be cultured in induction medium for 2 days to induce neuroectodermal differentiation. The spheroids with neuroectoderm are then embedded in Matrigel and cultured in expansion medium for 3-4 days to allow the formation of expanded neuroepithelial tissues. The spheroids are further cultured in maturation medium up to 60 days for neuronal differentiation and maturation to become mature HCOs (FIG. 12). A similar organization of PHOX2B-expressing domains is found in the day 60 control HCOs. As shown in FIG. 5B, the A5 domain (PHOX2B⁺; TH⁺; VGLUT2⁻) and C1 domain (PHOX2B⁺; TH⁺; VGLUT2⁺) are found and the RTN-like neurons (PHOX2B⁺; VGLUT2⁺; TH⁻) are residing next to the CI domain-like region. Moreover, 5-HT-expressing neurons are found in adjacent to these PHOX2B-expressing domains. In sum, these data indicate that PHOX2B⁺ neurons in HCOs retain their position code and can be assembled as a respiratory center-like region in the day 60 control HCOs, recapitulating their in vivo development.

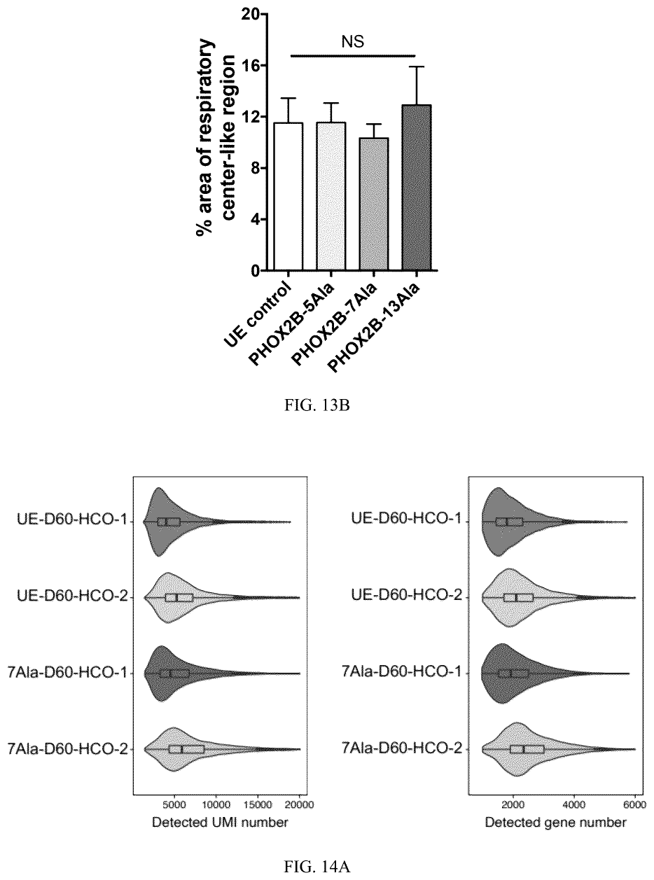

To examine how PHOX2B-PARMs affect the formation of respiratory center, three PHOX2B-PARM hPSC lines are used to make HCOs using the same differentiation protocol. It is found that the general morphology and the sizes of the day 60 HCOs derived from all the PHOX2B-PARM hPSC lines are highly comparable to that of the control (FIG. 5C). The regional diversities of the HCOs derived from the control and all PHOX2B-PARM hPSC lines are confirmed using immunostaining based on the expressions of various regional identity markers, where the control and all PHOX2B-PARM HCOs comprised forebrain (TBR1⁺), midbrain (FOXA2⁺) or hindbrain (ISL1⁺) regions (data not shown). Unlike in HBSO physical model, the cells expressing high level of PHOX2B detected in the mutant HCOs is much more. In both the control and mutant HCOs, PHOX2B⁺ cells are largely localized in the hindbrain (ISL1⁺) region and clustered near the 5-HT⁺ neurons to form respiratory center-like regions of comparable sizes (FIGS. 13A-13B). In all the PHOX2B-PARM HCOs, the A5 domain-like (PHOX2B⁺; TH⁺; VGLUT2⁻) regions can be identified, similar to the control HCOs, but the C1 domain-like (PHOX2B⁺; TH⁺; VGLUT2⁺) regions are absent in the HCOs derived from the PHOX2B-7Ala and PHOX2B-13Ala mutant lines (FIG. 5D). In the PHOX2B-5Ala HCOs, C1 domain-like region can be identified but the percentage of PHOX2B⁺ glutamatergic (PHOX2B⁺; VGLUT2⁺; TH⁻) RTN-like neurons are slightly reduced when compared to that of the control HCOs (FIG. 5D). Within the respiratory center-like regions, the numbers of PHOX2B-expressing cells are comparable between the control and all the PHOX2B-PARMs mutant HCOs (FIG. 5E), but the percentages of PHOX2B⁺; VGLUT2⁺double-positive cells are significantly lower in the PHOX2B-7Ala and PHOX2B-13Ala HCOs than that in the control HCOs (FIG. 5F). These demonstrate that PARMs in PHOX2B disrupt the formation of PHOX2B⁺; VGLUT2⁺ RTN neurons, which is consistent with the detriment effect of PHOX2B-7Ala in formation of glutamatergic PHOX2B⁺ neurons as seen in the HBSOs. In addition, with the HCO physical model, the impact of various PARMs in the formation of C1 neurons and the direct correlation between the severity of the defect with the length of the PARMs in PHOX2B, recapitulating the phenotypes as observed in CCHS-patients, are further illustrated.

### Example 6. PARM Interrupts the Progenitor-to-Neuron Transition of PHOX2B⁺ Cells in HCOs

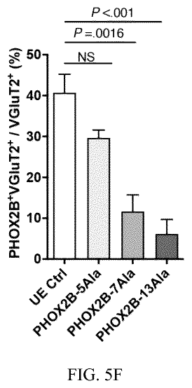

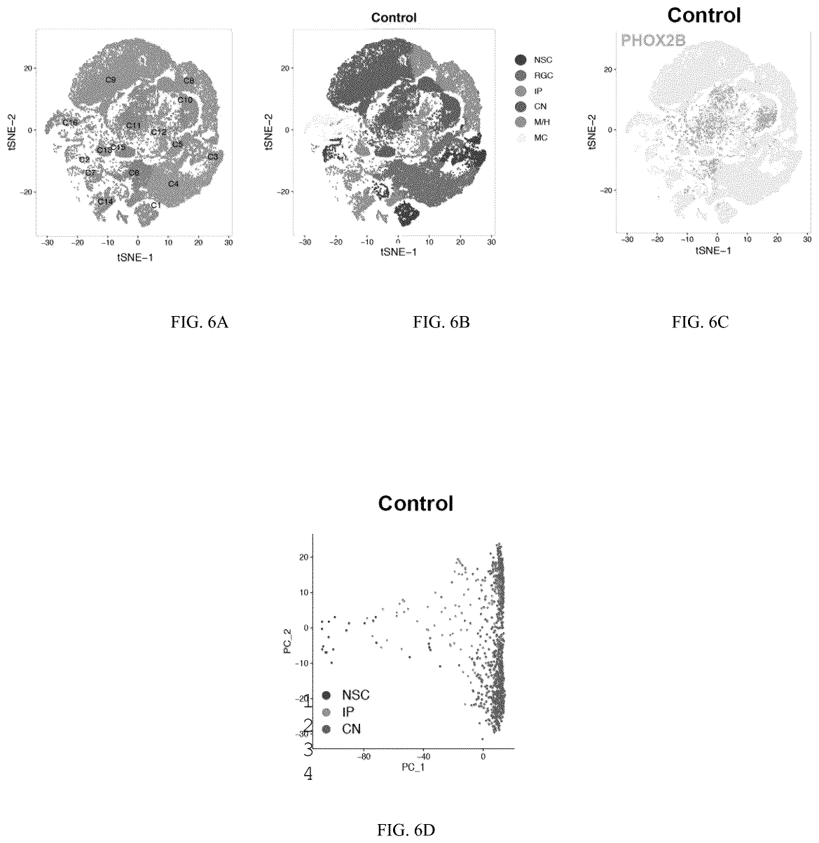

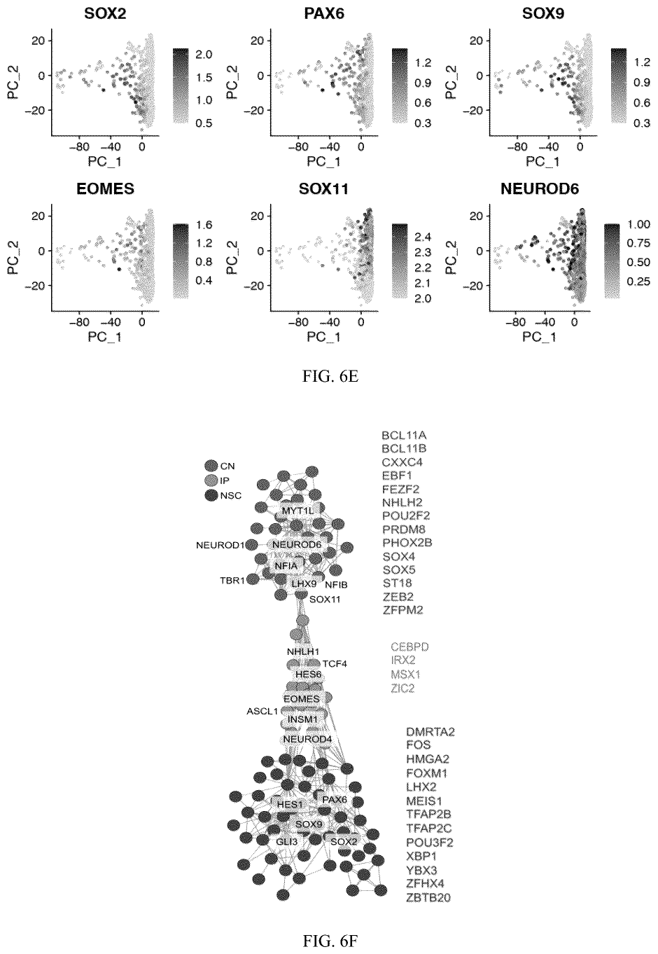

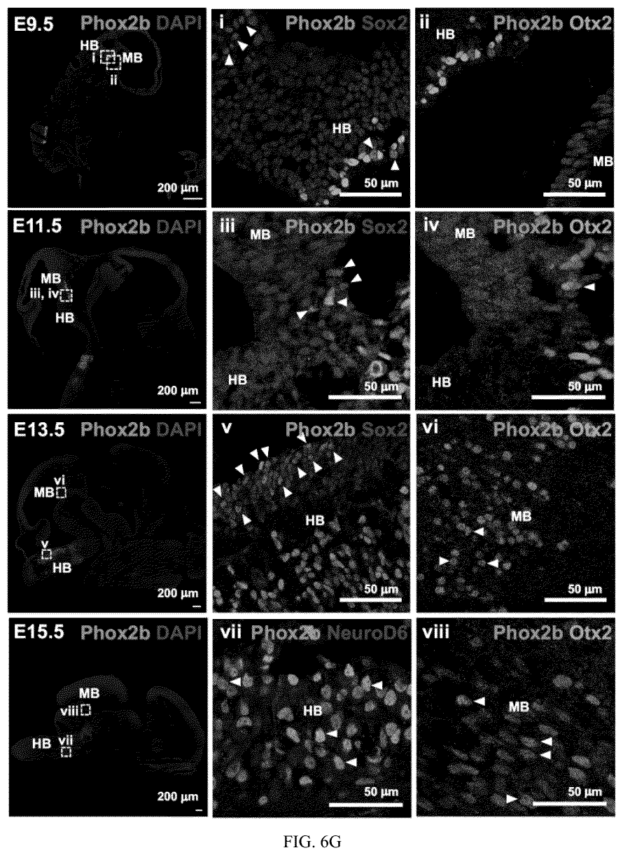

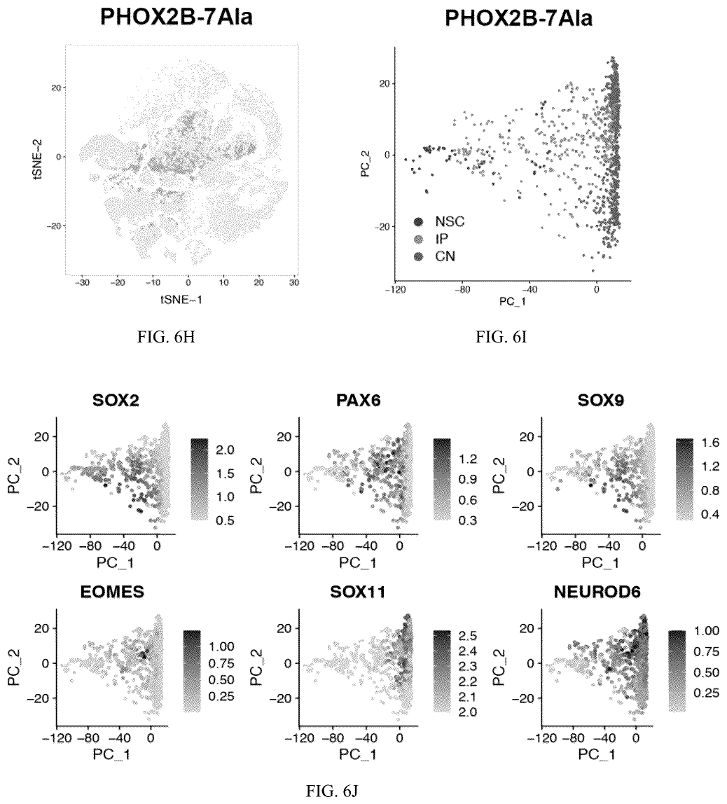

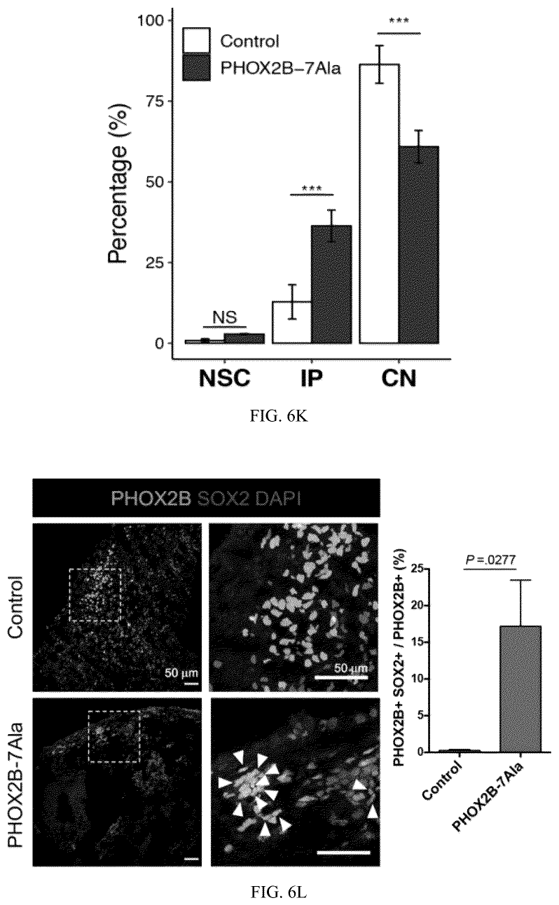

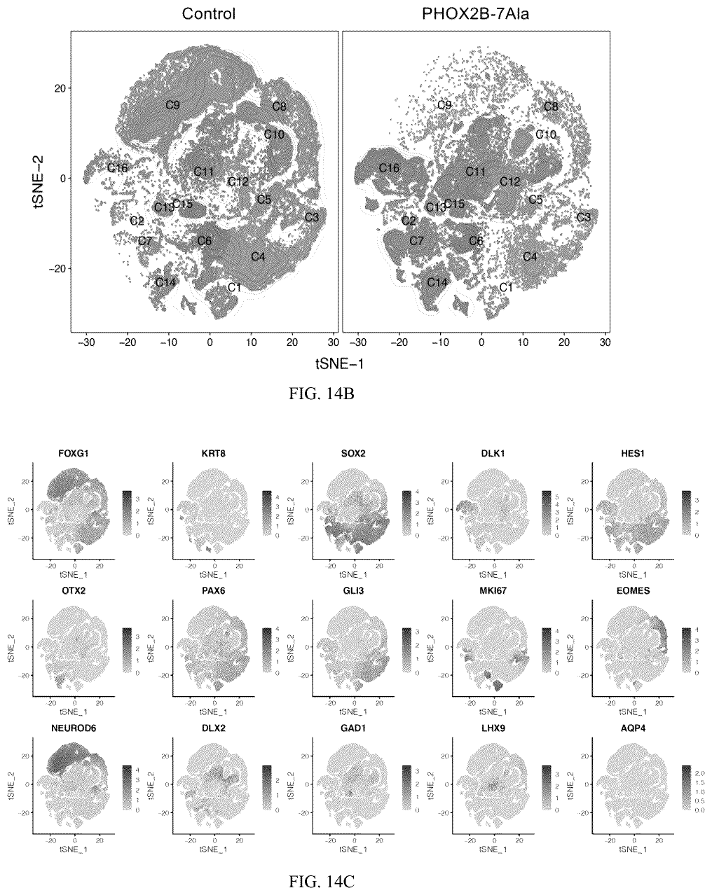

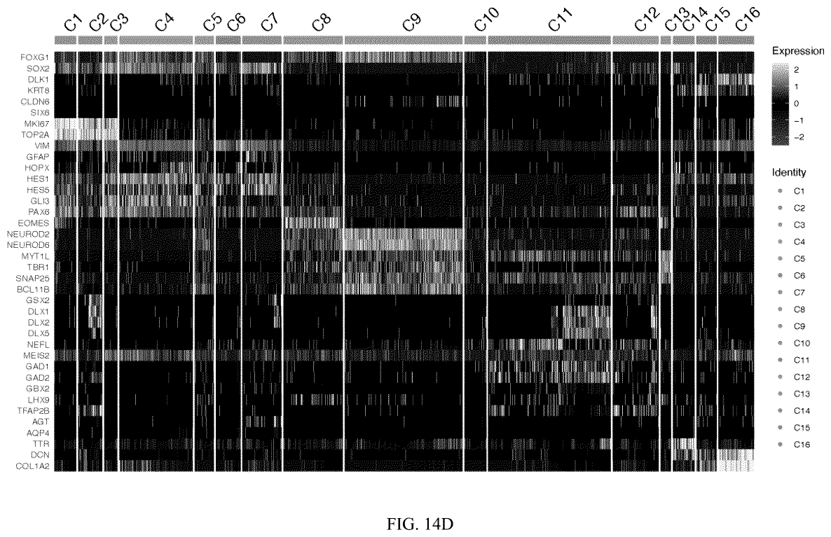

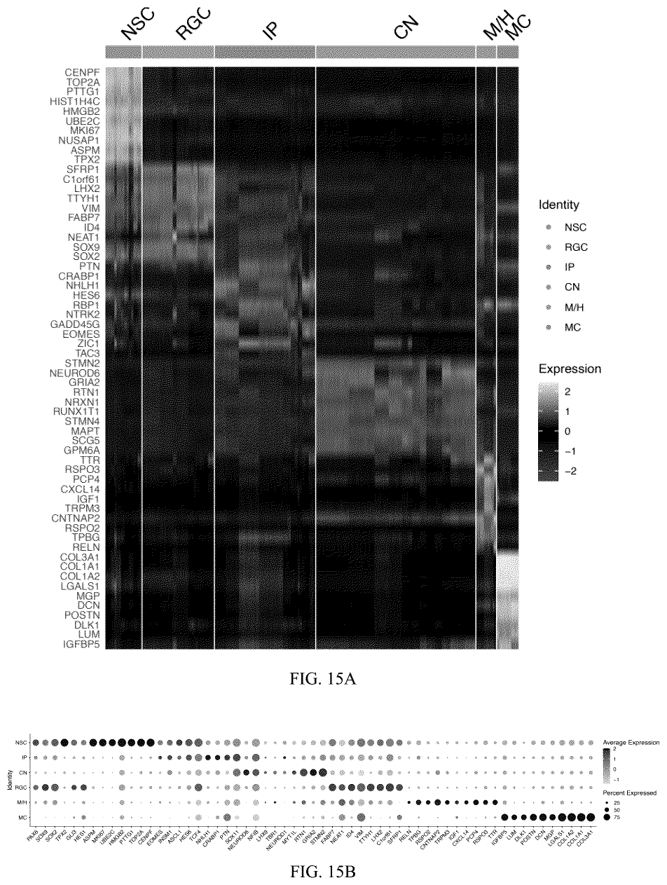

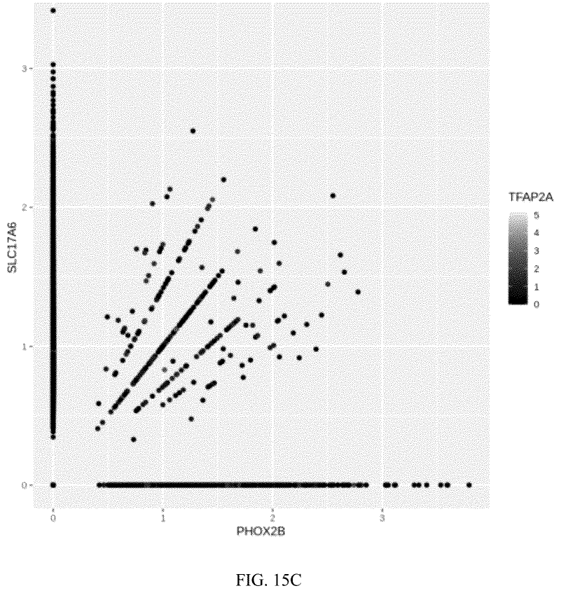

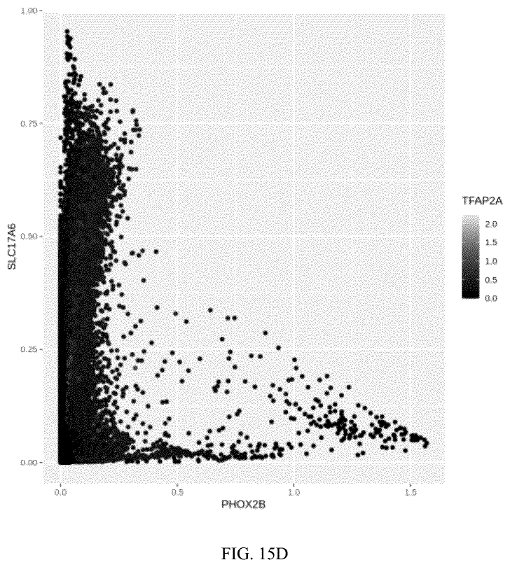

More PHOX2B⁺ cells are found in PHOX2B-7Ala HCOs when compared to the PHOX2B-7Ala HBSOs. Therefore, it is aimed to define the transcriptomic alternations associated with the expression of PHOX2B-7Ala. Cells from the day 60 control and PHOX2B-7Ala HCOs are sequenced at high resolutions using 10× Genomics to identify the major changes in gene expression dynamics associated with the PARMs in PHOX2B. A total of 82,564 individual cells are sequenced and 72,800 high-quality cells are obtained after quality control, 2,210 average genes as well as 20,370 mean confidently mapped reads per cell are identified (FIG. 14A). All single cells are projected on a t-distributed stochastic neighbor embedding (1-SNE) plot (FIG. 6A and FIGS. 14B-14D) and identified sixteen transcriptionally distinct clusters. The HCO cells of the present invention is overlaid with the 2-month old HCOs from public datasets where a similar differentiation protocol is used and removed potential technical and batch effects using Seurat software. The sixteen unbiased clusters are then further classified into six cell types, including neural stem cells (NSC), radial glia cells (RGC), intermediate progenitors (IP), cortical neurons (CN), midbrain/hindbrain (M/H) and mesenchymal-like cells (MC) (FIG. 6B and FIGS. 15A-15B) based on the expression of canonical markers and the cell annotation from the public datasets.

Unexpectedly, the expression of PHOX2B is extremely low in HCO cells (FIG. 15C), which may be due to the high cell heterogeneity in HCOs and that leads to a high dropout rate of the rare cells in 10× Genomics platform, making most of the PHOX2B of the relative short transcript length undetectable. Thus, a method using Markov affinity-based graph imputation of cells (MAGIC) is applied to denoise the cell count matrix and fill in missing transcripts. The expression of PHOX2B is well-recovered (FIG. 15D). Most of the PHOX2B⁺ cells are found in cell clusters inferred to be less mature progenitors (IP) or more mature neurons (CN), and a few fell into NSC in control HCOs (FIG. 6C). The lineage relationship among the PHOX2B⁺ cells in control HCOs is next inferred using principal component analysis (PCA). The transition states that linked NSC, through IP to CN can be well-represented by PCI, while the diversity of CN is reflected on PC2 (FIG. 6D). Notably, the genes known to be enriched in NSC (e.g., SOX2), IP (e.g., EOMES), and CN (e.g., NEUROD6) exhibit unique expression patterns along the lineage path in the PCA plot. A small portion of PHOX2B⁺ cells stays at the beginning of PCI axis and expresses SOX2, PAX6, SOX9 and EOMES, indicating that they remained at the earlier cell states (NSC/IP) of the differentiation path. On the other hand, the majority of the PHOX2B⁺ cells are found at the end of PCI axis with higher expression of mature neuronal markers (SOX11 & NEUROD6), suggesting that most of PHOX2B⁺ cells are mature neurons (CN) in the control HCOs (FIG. 6E).

To further examine the transcriptional regulation underpinning the lineage differentiation of PHOX2B⁺ cells along the NSC-IP-CN path, a TF correlation network (FIG. 6F) is constructed. The network analysis reveals three highly-interacted TF subnetworks regulating cells at NSC, IP and CN states. This unbiased approach can identify the master regulators of each cell state by locating genes at the central of each subnetwork. For example, PAX6 and SOX2 are tightly connected with other genes in the subnetwork and highly expressed in most of the NSCs, so they are considered as the core genes for maintaining cells at the neural stem cell state. ASCL1 and EOMES are the two core genes in IP state and their expressions are in concomitant with the NSC-to-IP transition, consistent with their roles in delamination and early neuronal specification. MYTL1 and NEUROD6 are found in another tightly connected subnetwork corresponding to mature neurons, and they are related to neuronal differentiation and their expressions are maintained at high level in mature neurons. These largely confirm that the current knowledge of transcriptional regulation during cortical neurogenesis can be applied to PHOX2B⁺ cells and PHOX2B⁺ cells likely follow a similar differentiation path as the other neuronal cells found in HCOs as described previously.

It is further elucidated whether the inferred differentiation trajectory of PHOX2B⁺ cells exists in vivo during the brain development, by examining the expressions of the cell-stage-specific markers of NSC/IP (Sox2) and CN (NeuroD6) with Phox2b in embryonic mouse brains at various developmental stages (E9.5, E11.5, E13.5 & E15.5). As shown in FIG. 6G, the Phox2b⁺ cells at NSC/IP state (Phox2b⁺; Sox2⁺) is found in the ventral hindbrain at E9.5 and in the ventral side of mid-hindbrain boundary (as marked by Otx2) at E11.5. At E13.5, some Phox2b⁺; Sox2⁺progenitor cells are residing at the dorsal pial surface of the hindbrain and Otx2⁺midbrain/hindbrain boundary, while they become undetectable at E15.5. From E15.5 onwards, Phox2b⁺ cells start expressing NeuroD6, suggesting that they are committed and proceeded to a more advanced/mature cell state. This demonstrated that the in vivo development of PHOX2B⁺ cells can be nicely recapitulated in HCOs at both cellular and molecular levels.

A comparative study with PHOX2B-7Ala HCOs is therefore used to delineate how PHOX2B-7Ala mutation may alter the NSC-IP-CN lineage differentiation of PHOX2B⁺ cells. Even comparable numbers of PHOX2B⁺ cells (FIG. 6H) and similar differentiation trajectory (FIGS. 61 and 6J) are detected in PHOX2B-7Ala HCOs after imputation, the distribution of PHOX2B⁺ cells among cell types is quite different from that in the control HCOs. Percentage of PHOX2B⁺ cells at CN state is significantly reduced while significantly more PHOX2B⁺ cells are staying in the progenitor (IP) state in PHOX2B-7Ala HCOs (FIG. 6K). Subsequent immunofluorescence analyses of the day 60 HCOs further confirm the higher percentage of PHOX2B⁺ cells at NSC/IP state (PHOX2B⁺; SOX2⁺) (FIG. 6L), accompanied by a reduced percentage of mature PHOX2B⁺ neurons (PHOX2B⁺; NEUROD6⁺) (FIG. 6M) in the PHOX2B-7Ala HCOs when compared to the control. With the HCO physical model, it is demonstrated that 7Ala-PARM in PHOX2B interrupts the progenitor-to-neuron transition during the development of PHOX2B⁺ neurons.

### Example 7. Dysregulation of Pattern Specification Pathway Genes and Loss of PHOX2B⁺ Neurons in OTX2⁺Domain of PHOX2B-7Ala HCOs

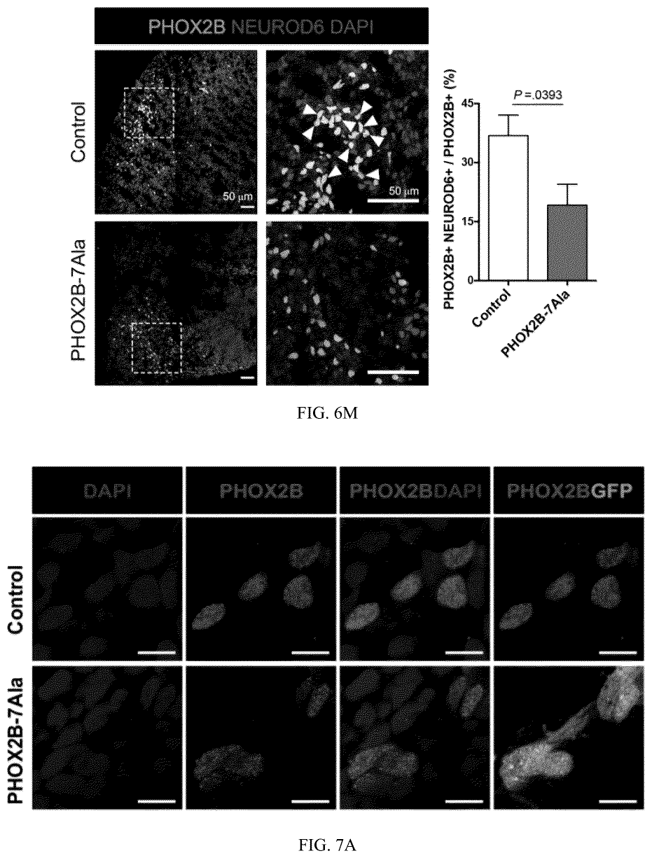

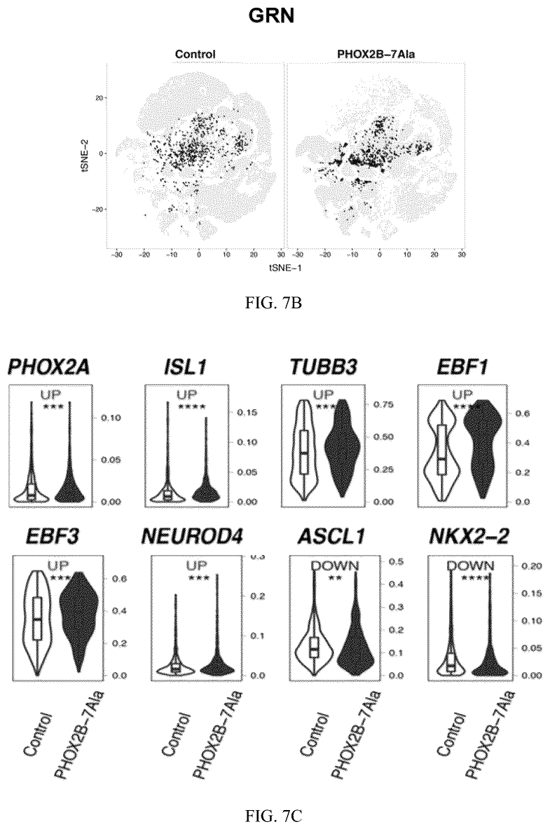

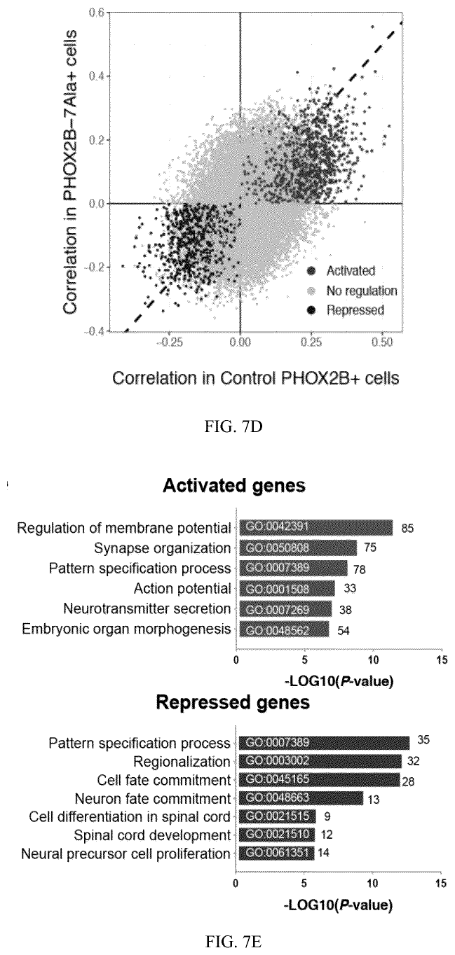

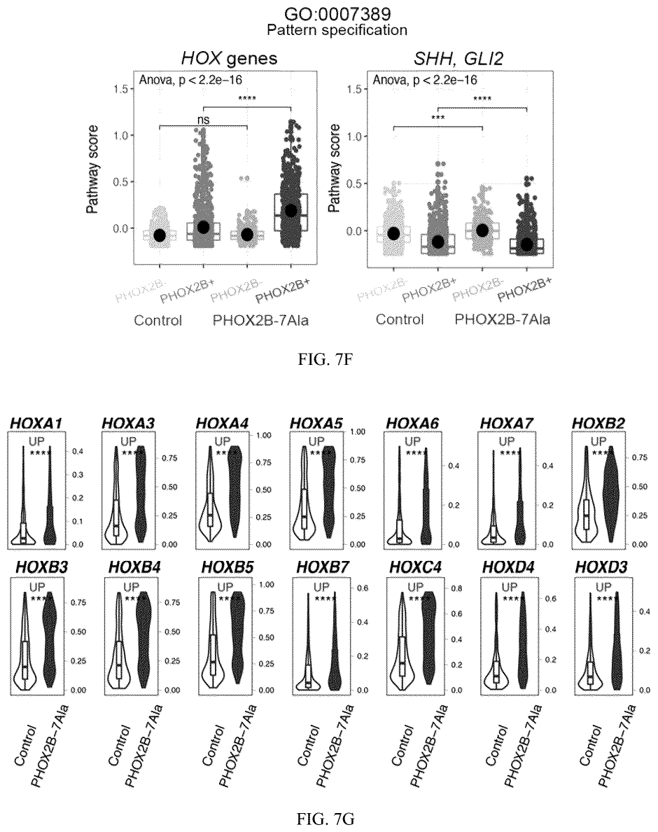

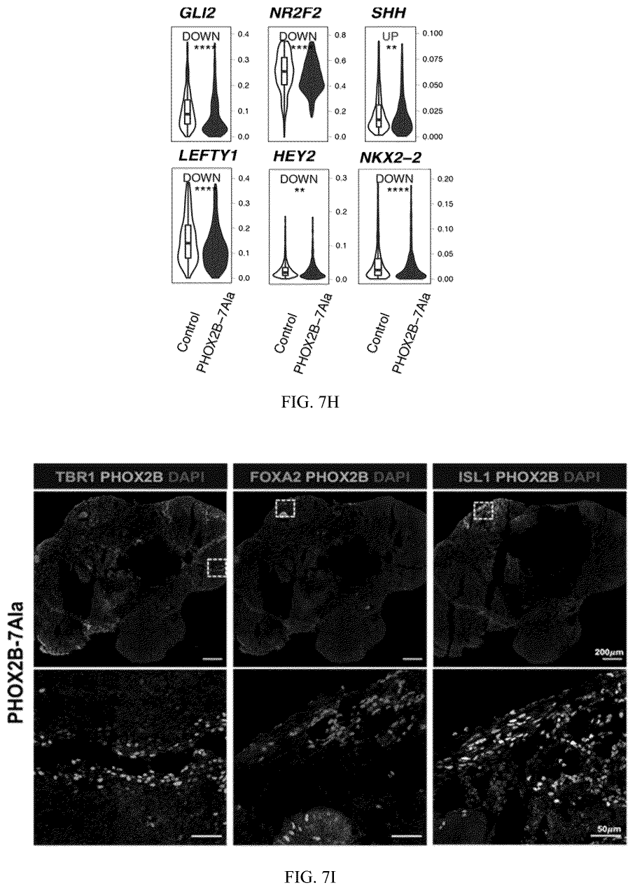

It has been suggested PARMs cause cytoplasmic retention of PHOX2B and thus disrupt the transcriptional activity of PHOX2B. When the subcellular localization of PHOX2B in the HCO neurons are examined, it is found that both control and mutant PHOX2B proteins are localized in the nuclei of the neurons in HCOs, where expression of PHOX2B-7Ala mutant allele is confirmed by co-expression of GFP signals (FIG. 7A). The results indicate that PHOX2B-7Ala does not cause cytoplasmic retention of PHOX2B proteins in the CNS neurons. Therefore, the Single Cell Regulatory Network Inference and Clustering (SCENIC) analysis is performed to establish a gene regulatory network (GRN) of PHOX2B for cells in control and mutant HCOs to further explore the gene network that regulated by PHOX2B-7Ala. PHOX2B-7Ala cells show different regulatory architecture compared to that in the control HCOs (FIG. 7B). Genetic ablation of Phox2b in mouse discovered the key genes implicated in differentiation events subsequent to the onset of Phox2b expression during normal hindbrain development, it includes up-regulation of Phox2a, Isl1, Tubb3, Ebf1-3 and down-regulation of Ascl1 and Nkx2.2. A similar expression pattern of these genes is observed in the cells expressing PHOX2B-7Ala, but greater changes are detected in the mutant cells when compared to the cells expressing the wild-type PHOX2B (FIG. 7C). Therefore, it is conceivable that PHOX2B-7Ala does not simply lead to loss-of-function or function in dominant negative manner, while it may cause aberrant expression of different gene sets. To address this point, a whole-transcriptome correlation analysis is performed to identify PHOX2B-7Ala-mediated (activated or repressed) genes and pathways using a 3-step approach. The correlation (co-expression) between PHOX2B and the other genes in the control and mutant HCOs is first evaluated to identify potential targets of wild-type and mutant PHOX2B. Secondly, the genes that are differentially regulated by the wild-type and mutant PHOX2B leading to the significant alterations in the regulatory architecture of PHOX2B among the control and mutant HCOs are assessed. Finally, to further identify potential direct target genes of PHOX2B-7Ala, a motif enrichment analysis is performed on the differentially expressed PHOX2B-associated genes between the control and mutant cells (based on an assumption that the wild-type and PHOX2B-7Ala recognize the same binding motif sequence). From these analyses, 2,393 and 640 genes are identified as the PHOX2B-7Ala activated (red dots) and repressed (blue dots) genes, respectively, where the activated genes represent the direct targets of the PHOX2B-7Ala with binding motif(s) in their promoter regions (FIG. 7D). Based on Gene Ontology (GO) database, the putative PHOX2B-7Ala activated/repressed genes are mainly implicated in pattern specification or neurogenesis (FIG. 7E). Intriguingly, HOX− and Hedgehog pathway (SHH, GLI2)-associated genes belonging to GO:0007389 (pattern specification process) are found significantly up- and down-regulated, respectively, in the PHOX2B-7Ala expressing cells, suggesting this pathway has been severely interrupted in the mutant cells. To evaluate how different genes belonging to this pathway are involved in PHOX2B⁻ and PHOX2B⁺ cells in control and mutant HCOs, the overall changes of these two pathway gene sets are further scored using an additive model. From these analyses, the up-regulation of HOX− and the down-regulation of Hedgehog pathway-associated genes implicated in the pattern specification are found in the PHOX2B⁺ cells, but their log₂ fold changes (PHOX2B⁺/PHOX2B⁻) are much higher in the mutant (FIG. 7F), including up-regulation of a panel of HOX genes (FIG. 7G) and down-regulation of multiple downstream effectors of SHH (FIG. 7H). Noteworthy, the Hedgehog pathway-associated genes, but not HOX-associated gene set, are slightly affected in PHOX2B⁻ cells in the PHOX2B-7Ala mutant HCOs (FIG. 7F), suggesting that the patterning defects in PHOX2B⁺ may also influence the neighboring PHOX2B⁻ cells and it further evidences the non-cell autonomous effect of PHOX2B-7Ala in hindbrain development. In sum, these analyses suggest that 7Ala-PARM in PHOX2B likely alter its transcription activity, leading to either overly activation or down-regulation of its target genes.

In line with the in silico predictions, the region-specific localization of PHOX2B⁺ cells is found to be affected in the HCOs with 7Ala-PARM in PHOX2B. Although the some of PHOX2B⁺ cells still retain the hindbrain identity and are localized in the ISL1⁺hindbrain regions, but not in the FOXA2⁺midbrain domains nor the TBR1⁺forebrain domains of the HCOs in PHOX2B-7Ala HCOs (FIG. 7I), they might have lost some of patterning information. For instance, in control HCOs, some PHOX2B⁺ cells are located in OTX2⁺domain of the mid-hindbrain boundary (MHB), but it is not observed in the mutant HCOs (FIG. 7J), implying that the genetic program for the positioning of PHOX2B⁺ cells may be altered in the mutant HCOs. Phox2b has been suggested to regulate the dopaminergic neuron development in the embryonic mouse midbrain, so PHOX2B-7Ala may also interrupt the formation of dopaminergic neurons in the MHB. Taken together, it is found that the pattern specification genes are severely interrupted in the mutant PHOX2B⁺ cells, with a concomitant loss of PHOX2B⁺ cells in the respiratory center and the OTX2⁺domain in the PHOX2B-7Ala HCOs.

### Example 8. PHOX2B-PARMs-Associated CNS and ENS Defects as Revealed in hPSC-Based Physical Models of CCHS-HSCR

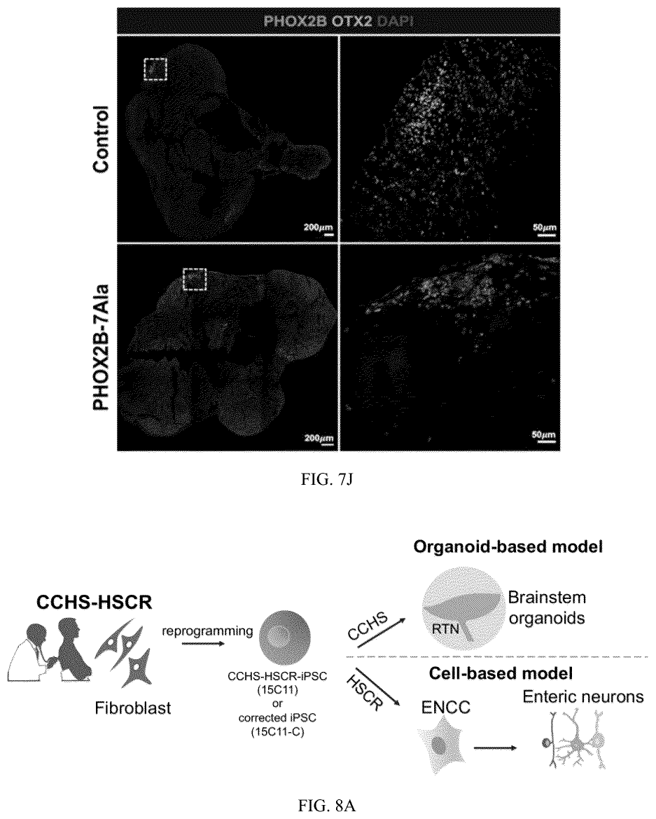

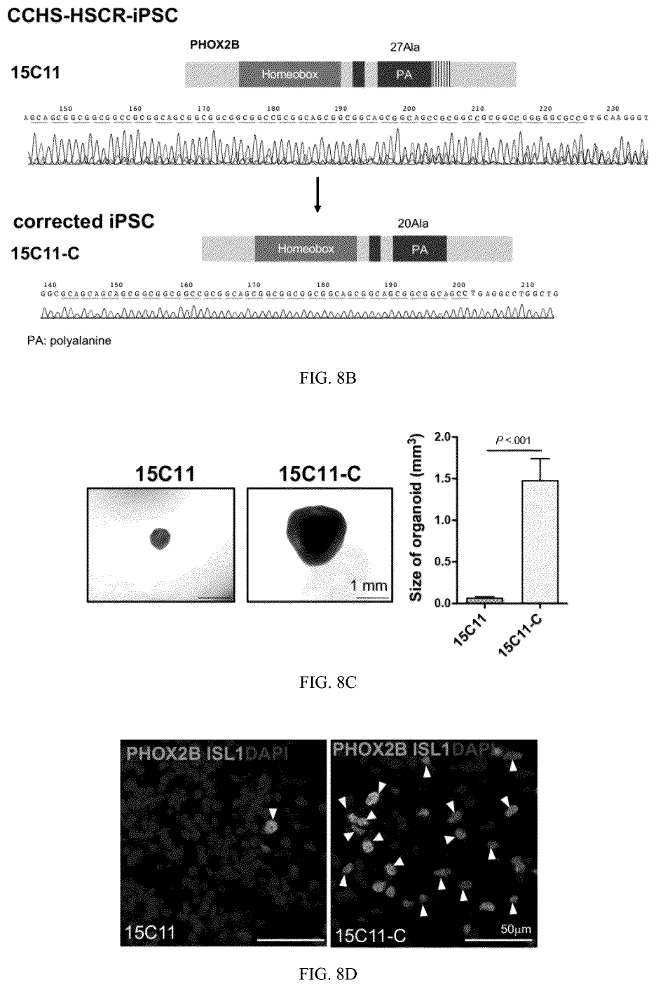

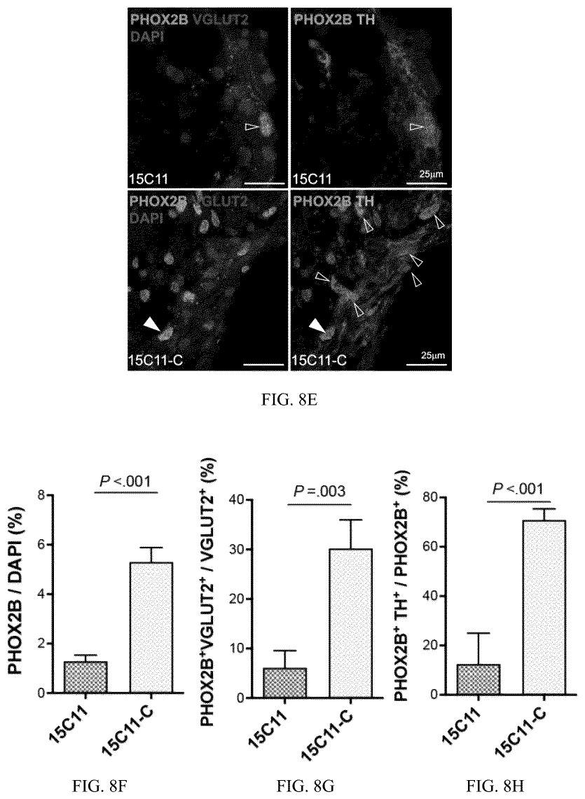

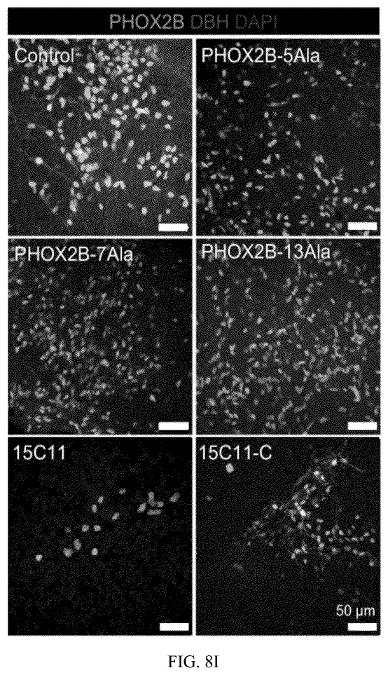

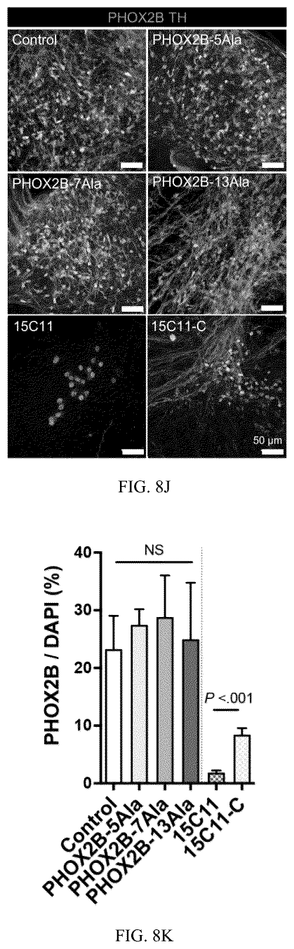

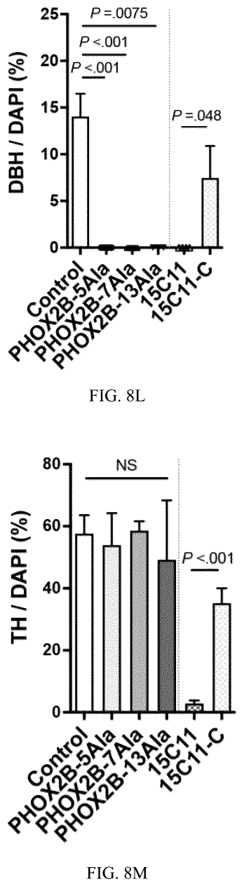

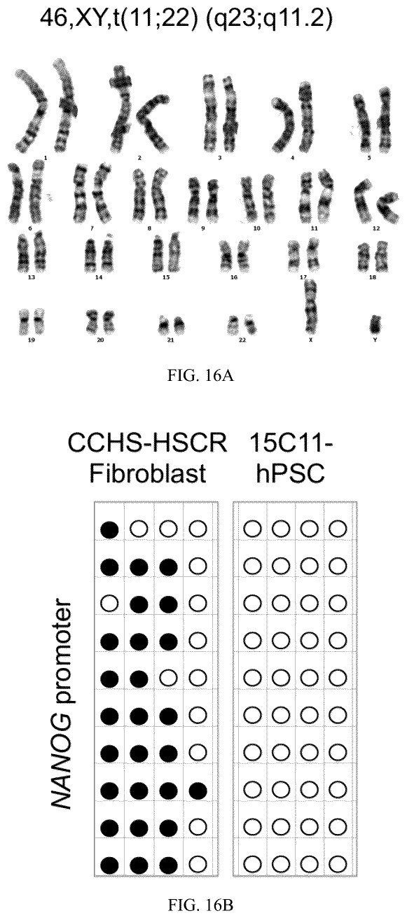

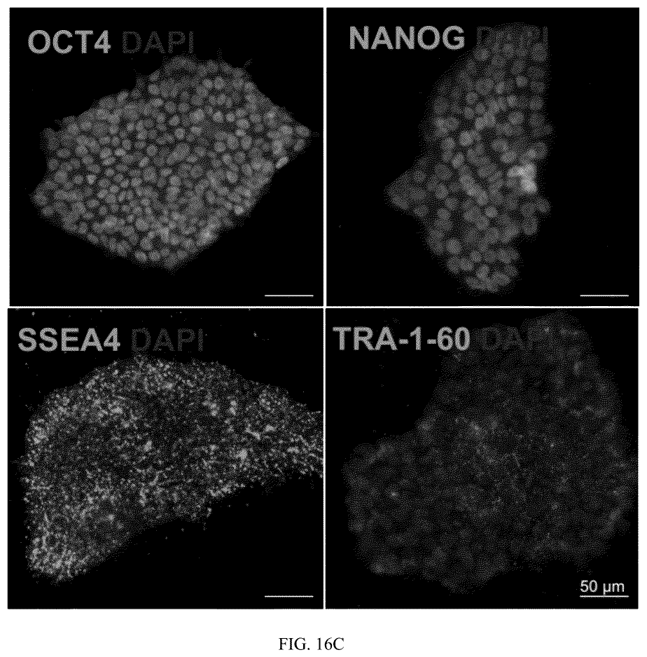

The HBSOs are consistently enriched with PHOX2B⁺ RTN-like neurons. Therefore, the HBSO physical model is further used to establish the causal link between PHOX2B-PARMs with CCHS. A patient-specific hPSC line is established using skin biopsy from a patient with CCHS and HSCR who carries a heterozygous +7Ala repeat mutation in the PHOX2B gene. In brief, skin fibroblast is obtained from the patient, reprogrammed with the episomal reprogramming vectors carrying the 4 reprogramming factors (Oct4, Klf4, Sox2 and c-Myc) to establish a CCHS-HSCR-hPSC line (15C11) (FIG. 8A and FIGS. 16A-16C). Clinically, the CCHS-HSCR patients have the clinical features of CCHS, short-HSCR, Marcus gunn phenomenon, and global developmental delay and show the genotype of PHOX2B-7Ala-PARM, in which t(11;22) (q23;q11.2).

For the subsequent evaluation of the weight of the 7Ala-PARM in causing CCHS and HSCR conditions, a “corrected” line (15C11-C) is generated, where the heterozygous +7Ala repeat mutation in the PHOX2B locus is “corrected” using CRISPR-Cas9-mediated HDR (FIG. 10). Briefly, the strategy for the correction of patient-specific PHOX2B mutation is similar to the protocol described above except for the use of the wild-type PHOX2B donor plasmid instead of the mutant PHOX2B-PARM donor plasmids. In brief, CCHS-HSCR (15C11) hPSCs are transfected with the wild-type PHOX2B donor plasmid, the gRNA constructs and a Cas9-D10A nickase expression plasmid using Human Stem Cell Nucleofector Kit 2. Selection and screening of positive clones are performed as described above. The presence of heterozygous +7Ala repeat mutation in the patient-specific hPSC line and the successful “correction” of PHOX2B gene in the “corrected” line are confirmed by Sanger sequencing (FIG. 8B).

Surprisingly, the overall size of the day 60 HBSOs derived from 15C11 hPSC line is very small, while the size of HBSOs become more comparable to those generated by the control line when the heterozygous +7Ala repeat mutation in the patient line is corrected (FIG. 8C). 15C11 HBSOs have similar defects as observed in the PHOX2B-7Ala HBSOs, including the loss of PHOX2B⁺ (FIG. 8D), PHOX2B⁺TH⁺ and PHOX2B⁺VGLUT2⁺ neurons (FIG. 8E), and these defects are significantly rescued in the 15C11-C HBSOs (FIGS. 8F-8H), suggesting that the defects associated with CCHS are 7Ala-PARM-dependent.

The ENS-associated defects are modeled using a directed differentiation protocol. In this protocol, human pluripotent stem cells (hPSCs) are induced to neural crest cells as previously described. Briefly, hPSCs are dissociated into a single-cell suspension by treating with Accutase (STEMCELL Technologies, 07922) for 10 minutes at 37° C. and seeded on a Matrigel-coated culture dish at a density of 4-5×10⁴ cells cm-2 in iPS medium (DMEM/F-12 with GlutaMAX supplement, 20% (v/v) KnockOut Serum Replacement (KOSR), 1 mM L-Glutamine, 1% NEAA, 1% β-mercaptoethanol, and 0.5% Pen-Strep) supplemented with FGF-2 (10 ng/ml) and Y-27632 (10 μM). The day following cell seeding is marked as day 0.

Neural crest differentiation is initiated by replacing iPS medium with KSR medium (KnockOut DMEM, 15% (v/v) KOSR, 2 mM L-Glutamine, 1% (v/v) NEAA, 1% (v/v) β-mercaptocthanol, and 1% (v/v) Pen-Strep) at day 0 and gradually switching to N2 medium (DMEM/F-12 with GlutaMAX supplement and Neurobasal Medium in a 1:1 ratio, supplemented with 0.5% (v/v) B-27 Supplement, 0.5% (v/v)N-2 Supplement, 5 mg/ml insulin, and 1% (v/v) Pen-Strep) from day 4 to 9 (KSR medium ratio=3:1 (day 4 to 5); 1:1 (day 6 to 7); 1:3 (day 8 to 9)). To induce the neural crest cells to enteric lineage (hENCCs), the culture media are supplemented with LDN-193189 (100 nM) from day 0 to 2, SB431542 (10 μM) from day 0 to 3, WNT activator CHIR99021 (3 μM) from day 2 to 9, and RA (1 μM) from day 6 to 9. The culture medium is changed daily.

The FACS-enriched hENCCs are then cultured in neuronal medium to generate a diversity of neuronal derivatives (hereafter, named as ENS neurons) expressing various neuronal markers. PHOX2B-PARMs (PHOX2B-5Ala, PHOX2B-7Ala, and PHOX2B-13Ala) alone do not affect hENCC yield nor their subsequent neuronal differentiation to make PHOX2B⁺ neurons (FIGS. 8I and 8K), including TH⁺ (FIGS. 8J and 8M) ENS neurons, but they deter the formation of dopamine beta-hydroxylase (DBH) neurons where DBH is the direct target of PHOX2B (FIGS. 8I and 8L). On the other hand, the patient-hENCCs exhibit defects in making both DBH⁺ (FIG. 8I) and TH⁺ (FIG. 8J) neurons. Correction of PHOX2B-7Ala PARM in patient hPSC significantly increases the yield of these neurons (FIGS. 8I-8M). These data illustrate that PARMs in PHOX2B or the loss of a copy of the wild-type PHOX2B gene likely interrupt the formation of DBH⁺ ENS neurons, but the ENS phenotypes of the patient are not attributed solely to the PHOX2B-7Ala-PARM, where additional mutation(s) or sensitizing genetic background are likely involved.

Overall, various tools (PHOX2B-PARMs and CCHS-HSCR patient specific-hPSC lines) and the experimental paradigms (use of regionalized (HBSOs) and unguided (HCOs) organoids) are established in the present invention for assessing the molecular controls underpinning the formation of the hypercapnia-responsive domain of respiratory center and the molecular basis of CCHS-HSCR using hPSC-based physical models.

A robust differentiation protocol is present to generate HBSOs which contains a diversity of CNS neurons with hindbrain signatures. More importantly, the respiratory center-like region composed of PHOX2B⁺ glutamatergic RTN neurons, the A5 (PHOX2B⁺VGULT2⁻ TH⁺), C1 (PHOX2B⁺VGULT2⁺ TH⁺)-like neurons and the afferent inputs of 5-HT⁺ neurons could be identified in the HBSOs, with functional response to hypercapnia. Subsequent high-resolution analysis of the transcriptomes of cells from the control and PHOX2B-7Ala PARM HBSOs, the cell-autonomous and non-cell autonomous effects of PHOX2B-7Ala PARM on generation of RTN neurons and the respiratory center are further demonstrated, where PHOX2B-7Ala deters not only the formation of the glutamatergic RTN neurons, but also the differentiation of other lineages of hindbrain neurons, perturbing the cell composition of HBSOs.

To unbiasedly reveal the molecular mechanisms underlying the cell-autonomous and non-cell autonomous effects of PHOX2B-7Ala PARM in the formation of the respiratory center and its implications in CCHS pathogenesis, PHOX2B⁺ cells in control and mutant HCOs where various regions of brain are self-patterned are analyzed. The results suggest that HCOs recapitulate the formation of RTN-respiratory center, where PHOX2B⁺VGULT2⁺TH⁻ RTN-like neurons can be self-assembled with other functional domains of respiratory center including the Cl and A5 domains, with a cytoarchitecture highly resembling the RTN-respiratory center. Using various PHOX2B-PARM hPSC lines, it is further demonstrated that HCOs can phenocopy the mouse mutant of Phox2b-7Ala with the defects in formation of RTN neurons in the respiratory center-like region. Subsequent scRNA-seq analysis further defines the differentiation trajectories of PHOX2B⁺ neurons and the underlying GRNs. A very similar differentiation trajectory of PHOX2B⁺ neurons is observed between the HCOs and the developing mouse brains, where PHOX2B is co-expressed with progenitor (NSC/IP) and mature neuronal (CN) markers, indicating that PHOX2B expression has started in an early stage of neuronal differentiation and persists in the mature neurons as described previously. Furthermore, as demonstrated by the unbiased TF correlation network analysis, these PHOX2B⁺progenitors likely go through similar differentiation paths as other CNS lineages during the brain development. More intriguingly, PHOX2B⁺ neurons retain the hindbrain signatures with specific migratory path, destination and identity. In HCOs, PARM in PHOX2B, not only interferes the progenitor-to-neuronal transition of PHOX2B⁺ neurons along their neuronal differentiation, but also their morphogenesis and pattern specification. The GO analyses from the scRNA-seq analyses of the PHOX2B-7Ala mutant HCOs illustrate that PHOX2B-7Ala represses genes implicated in pattern specification, regionalization and neuronal migration, in addition to those genes associated with neuron fate differentiation. Particularly, many HOX genes are dysregulated in the PHOX2B-PARM HCOs. Since the HOX genes regulate the effectors of SHH signaling as well as ASCL1/NGN signaling during rostro-caudal and dorso-ventral patterning specification during early hindbrain development, PHOX2B-PARMs may actually disrupt the CNS development through dysregulating the SHH signaling. The defective patterning of PHOX2B⁺ neurons may account for their absence in the OTX⁺ domain and the RTN-respiratory center in the PHOX2B-PARM-HCOs and/or HBSOs. Concordant with these observations, in Phox2b-7Ala mouse mutants, the loss of RTN neurons is accompanied by a slight increase in the number of Phox2b; Vglut2 double-positive cells in the dorsal side of the facial nucleus (nVII), implying that the loss of RTN neurons in the ventral side of nVII may be caused by the failure of the Phox2b⁺Vglut2⁺ cells to migrate or position in a proper region in the hindbrain, thus affecting the neuronal circuitry for the control of respiration.

As demonstrated in the present invention, the complementary uses of HBSO− and HCO− models address a board range of scientific questions. For instance, HBSOs are enriched with hindbrain neurons and of high reproducibility, and that makes it most suitable for functional analyses of cell autonomous effect of PHOX2B-PARMs. The hypercapnia-responsive neurons in HBSOs can be easily detected not only based on the expression of the molecular markers, but also their electroactivities in response to the change in CO₂ levels, ion channel mediators or neurotransmitters. This greatly expedites its potential applications for the high throughput drug screenings. On the other hand, the HCOs are generated at a close-to-physiological condition and driven by the self-patterning signals, allowing the study of the interactions among various functional domains as well as genes implicated in the patterning of the brain, and the non-cell autonomous actions of PHOX2B-PARMs.

The ability to make CNS and PNS neurons of different cellular origins represents another major advantage of the use of hPSC. Despite of the high similarity of these two systems, CNS and PNS frequently exhibit different vulnerability to various disease-associated mutations. Here, CNS and enteric neurons from the same hPSC lines with identical genetic backgrounds are made and a side-by-side comparison is performed on the impacts of PARMs on CNS and ENS neurons. Surprisingly, it is found that the previously reported cytoplasmic retention of PHOX2B with PARMs longer than +9 Ala is not observed in the PHOX2B-13Ala mutant cells. The mislocalization of PHOX2B-PARM proteins is probably an artifact due to the overexpression of PHOX2B-PARMs performed in the previous studies. Based on the results in the CNS and ENS neurons, PHOX2B-PARM proteins, when it is expressed in a normal endogenous level, can still localize in the nuclei of the mutant cells, implying that the pathogenicity of PHOX2B-PARMs is not related to the protein mislocalization as described previously. On the other hand, the transcriptomic analysis suggests that PHOX2B-PARMs may actually have a toxic gain of function which alters its GRNs. In addition, we showed that PHOX2B-PARMs, regardless of their lengths, disrupt the formation of PHOX2B⁺VGLUT2⁺TH⁻ RTN and DBH⁺ enteric neurons in the hPSC-based CNS and ENS models, while PARMs of longer alanine repetition also perturb the formation of PHOX2B⁺VGLUT2⁺TH⁺ neurons in C1. It has been shown that the Cl neurons innervating to the A5 domain are important for sympathetic and respiratory outputs, so the additional loss of the Cl neurons associated with the longer polyalanine repetition in PARMs probably leads to more severe breathing defects.

Using a CCHS-HSCR patient specific- and a “corrected”-hPSC lines, the direct impacts of PHOX2B-PARMs on CCHS− and HSCR-associated phenotypes are further unveiled. It is consistent with the mouse models, PHOX2B-7Ala alone is sufficient to extirpate the RTN neurons in the HBSOs derived from the PHOX2B-PARM and patient-specific hPSC lines. On the other hand, in ENS, PARMs in PHOX2B specifically interrupt the formation of DBH⁺ neurons, while the generation of other enteric neurons remain largely not affected. However, patient specific-hPSC derived ENS progenitors fail to make PHOX2B⁺ enteric neurons and the correction of PARMs in PHOX2B only partially rescues the differentiation defects. These data suggest that the sensitized genetic background comprising additional variations in the genome may predispose the patient to HSCR. In particular, karyotyping analysis reveals that this patient carries a common constitutional translocation, t(11;22) (q23;q11). Balanced carriers are phenotypically normal, but chromosome 22q11 is a susceptibility locus for HSCR. Therefore, it is conceivable that the t(11;22) (q23;q11) translocation represents a potential risk factor making this CCHS patient more susceptible to HSCR.

In sum, various hPSC-based organoid- and cell-models are applied to address the biological impacts of PHOX2B-PARMs in CNS and ENS of different cellular origins. The present invention has highlighted the distinct and common entities of these two closely related nervous systems, accounting for the different vulnerability of CNS and ENS to PHOX2B-PARMs.

As used herein and not otherwise defined, the terms “substantially,” “substantial,” “approximately” and “about” are used to describe and account for small variations. When used in conjunction with an event or circumstance, the terms can encompass instances in which the event or circumstance occurs precisely as well as instances in which the event or circumstance occurs to a close approximation. For example, when used in conjunction with a numerical value, the terms can encompass a range of variation of less than or equal to +10% of that numerical value, such as less than or equal to +5%, less than or equal to +4%, less than or equal to +3%, less than or equal to +2%, less than or equal to +1%, less than or equal to +0.5%, less than or equal to +0.1%, or less than or equal to +0.05%.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A hypoventilation physical model, comprising a brainstem organoid and a cerebral organoid, wherein both of the brainstem organoid and the cerebral organoid are formed by pluripotent stem cells (PSCs) having a PHOX2B gene mutation.

2. The hypoventilation physical model of claim 1, wherein the PHOX2B gene mutation comprises a polyalanine repeat expansion mutation (PARM).

3. The hypoventilation physical model of claim 2, wherein the PARM comprises a 5-PARM, a 7-PARM and a 13-PARM.

4. The hypoventilation physical model of claim 2, wherein the PARM is happened on a 20-Ala polyalanine tract in exon 3 of the PHOX2B gene.

5. The hypoventilation physical model of claim 1, wherein the brainstem organoid is fabricated by:
suspending the PSCs into a single cell suspension;
seeding the single cell suspension to a microwell plate with a density of 8,000-10,000 cells per microwell and incubating them overnight to form spheroids;
transferring the spheroids to a low-attachment plate comprising a first culture medium supplemented with a BMP inhibitor and a TGF-β inhibitor in a ratio of approximately 1:8 to 1:12 followed by culturing;
changing the medium to a second culture medium supplemented with the BMP inhibitor, TGF-β inhibitor, and a SHH agonist followed by culturing under agitation;
changing the medium to the second culture medium supplemented with the SHH agonist, a WNT agonist and retinoic acid (RA) followed by incubation;
changing the medium to a third culture medium supplemented with BDNF, GDNF, dibutyryl-cAMP and an acid for incubation; and
collecting matured brainstem organoids.

6. The hypoventilation physical model of claim 1, wherein the cerebral organoid is generated by:
suspending the PSCs in an embryoid body formation medium with ROCK inhibitor to form a single cell suspension;
transferring the PSCs to a low-attachment plate for culturing for 6-14 hours to form embryoid bodies;
further culturing the embryoid bodies in the embryoid body formation medium for 36-56 hours;
transferring the embryoid bodies to an induction medium for an incubation of 36-60 hours to induce the formation of neuroepithelia;
embedding the embryoid bodies in a basement membrane matrix for further expansion;
changing the medium to a maturation medium for culturing under agitation; and
collecting matured cerebral organoids.

7. The hypoventilation physical model of claim 1, wherein the brainstem organoid comprises retrotrapezoid nucleus (RTN) neurons and the brainstem organoid exhibits increased firing in response to elevated CO₂ levels.

8. The hypoventilation physical model of claim 1, wherein the cerebral organoid comprises functional neurons expressing glutamatergic, GABAergic, cholinergic, and serotonergic markers.

9. The hypoventilation physical model of claim 1, further comprising a neuronal differentiation medium optimized for producing enteric nervous system (ENS) neurons.

10. A system for screening a potential drug candidate for treating hypoventilation based on ventilatory response CO₂ changes, comprising the hypoventilation physical model of claim 1 for mimicking the impaired ventilatory response to hypercarbia and hypoxemia.

11. A method of treating hypoventilation, comprising identifying a candidate drug using the system of claim 10 and administering an effective amount of the candidate drug to a subject in need thereof.

12. A hypoventilation physical model, comprising a brainstem organoid and a cerebral organoid, wherein the brainstem organoid includes PHOX2B⁺ neurons with reduced dopamine beta-hydroxylase (DBH) expression.

13. The hypoventilation physical model of claim 12, wherein the PHOX2B gene mutation is corrected using CRISPR/Cas9 genome editing to increase the yield of DBH⁺ neurons.

14. The hypoventilation physical model of claim 12, wherein the PHOX2B gene mutation is introduced via genome editing techniques.

15. The hypoventilation physical model of claim 12, further comprising peripheral neurons derived from human enteric neural crest cells (hENCCs).

16. The hypoventilation physical model of claim 15, wherein the hENCCs are differentiated to express PHOX2B and exhibit ENS-associated defects.
